(19) United States
(12) Patent Application Publication
(10) Pub. No.: US 2016/0158246 A1
(43) Pub. Date: Jun. 9, 2016

(54) METHOD OF TREATING DIFFUSE LARGE B-CELL LYMPHOMA (DLBCL) USING A BET-BROMODOMAIN INHIBITOR

(71) Applicant: ONCOETHIX GMBH, Luzern (CH)

(72) Inventors: Francesco Bertoni, Bellinozona (CH); Giorgio Inghirami, New York, NY (US)

(73) Assignee: ONCOETHIX GMBH, Luzern (CH)

(21) Appl. No.: 14/910,344

(22) PCT Filed: Aug. 6, 2014
(86) PCT No.: PCT/EP2014/002164

§ 371 Date: Feb. 5, 2016

Related U.S. Application Data
(60) Provisional application No. 61/909,703, filed on Nov. 27, 2013.
(60) Provisional application No. 61/862,772, filed on Aug. 6, 2013.
(60) Provisional application No. 61/862,752, filed on Aug. 6, 2013.

(51) Int. Cl. A61K 31/551; A61K 47/38

(52) U.S. Cl. CPC A61K 31/551; A61K 47/38

(57) ABSTRACT

A method of treating diffuse large B-cell lymphoma comprising administering to a patient a pharmaceutically acceptable amount of a composition comprising a thienotriazolodiazepine compound, said thienotriazolodiazepine compound being represented by Formula (I), wherein R₁ is alkyl having a carbon number of 1-4, R₂ is a hydrogen atom; a halogen atom; or alkyl having a carbon number of 1-4 optionally substituted by a halogen atom or a hydroxyl group, R₃ is a halogen atom; phenyl optionally substituted by a halogen atom, alkyl having a carbon number of 1-4, alkoxy having a carbon number of 1-4 or cyano; —NR₅—(CH₂)ₘ—R₆ wherein R₅ is a hydrogen atom or alkyl having a carbon number of 1-4, m is an integer of 0-4, and R₆ is phenyl or pyridyl optionally substituted by a halogen atom; or —NR₇—CO—(CH₂)ₙ—R₈ wherein R₇ is a hydrogen atom or alkyl having a carbon number of 1-4, n is an integer of 0-2, and R₈ is phenyl or pyridyl optionally substituted by a halogen atom, and R₄ is —(CH₂)ₐ—CO—NH—R₉ wherein a is an integer of 1-4, and R₉ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR₁₀ wherein b is an integer of 1-4, and R₁₀ is alkyl having a carbon number of 1-4, or a pharmaceutically acceptable salt thereof or a hydrate or solvate thereof, wherein the patient has activated B-cell diffuse large B-cell lymphoma.

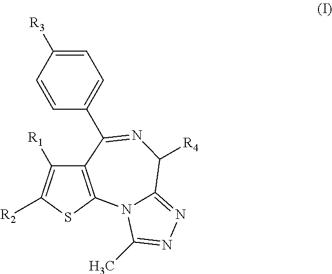

### CROSS-REFERENCE TO RELATED APPLICATIONS

This application claims the benefit of U.S. Provisional Application Ser. No. 61/862,752, filed Aug. 6, 2013, U.S. Provisional Application Ser. No. 61/862,772, filed Aug. 6, 2013, and U.S. Provisional Application Ser. No. 61/909,703, filed Nov. 27, 2013, each of which is incorporated herein by reference in its entirety.

### FIELD OF INVENTION

The present disclosure is concerned with methods of treatment, particularly methods of treating lymphoma in a mammal.

### BACKGROUND OF THE INVENTION

Epigenome deregulation in cancer cells affects transcription of oncogenes and tumor suppressor genes. BET Bromodomain proteins recognize chromatin modifications and act as epigenetic readers contributing to gene transcription. BET Bromodomain inhibitors have shown promising pre-clinical activity in hematological and solid tumors and are currently in phase I studies. The mechanism of action and relevant affected genes are not fully characterized and there are no established response predictors. We have shown activity of BET Bromodomain OTX015 in lymphoma cell lines.

### BRIEF SUMMARY OF THE INVENTION

In some embodiments, the present invention provides methods of treating diffuse large B-cell lymphoma comprising administering to a patient a pharmaceutically acceptable amount of a composition comprising a thienotriazolodiazepine compound, said thienotriazolodiazepine compound being represented by the following Formula (1):

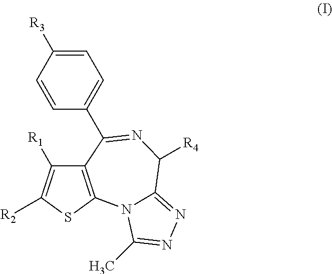

wherein R₁ is alkyl having a carbon number of 1-4, R₂ is a hydrogen atom; a halogen atom; or alkyl having a carbon number of 1-4 optionally substituted by a halogen atom or a hydroxyl group, R₃ is a halogen atom; phenyl optionally substituted by a halogen atom, alkyl having a carbon number of 1-4, alkoxy having a carbon number of 1-4 or cyano; —NR₅—(CH₂)ₘ—R₆ wherein R₅ is a hydrogen atom or alkyl having a carbon number of 1-4, m is an integer of 0-4, and R₆ is phenyl or pyridyl optionally substituted by a halogen atom; or —NR₇—CO—(CH₂)ₙ—R₈ wherein R₇ is a hydrogen atom or alkyl having a carbon number of 1-4, n is an integer of 0-2, and R₈ is phenyl or pyridyl optionally substituted by a halogen atom, and R₄ is —(CH₂)ₐ—CO—NH—R₉ wherein a is an integer of 1-4, and R₉ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR₁₀ wherein b is an integer of 1-4, and R₁₀ is alkyl having a carbon number of 1-4, or a pharmaceutically acceptable salt thereof or a hydrate or solvate thereof. In some embodiments, the patient has activated B-cell diffuse large B-cell lymphoma.

In some embodiments, the thienotriazolodiazepine compound represented by Formula 1 is independently selected from the group consisting of: (i) (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide or a dihydrate thereof, (ii) methyl (S)-{4-(3′-cyanobiphenyl-4-yl)-2,3,9-trimethyl-6H-thieno[3,2-f][1,2,4]tri-azolo[4,3-a][1,4]diazepin-6-yl}acetate, (iii) methyl (S)-{2,3,9-trimethyl-4-(4-phenylaminophenyl)-6H-thieno[3,2-f][1,2,4]triaz-olo[4,3-a][1,4]diazepin-6-yl}acetate; and (iv) methyl (S)-{2,3,9-trimethyl-4-[4-(3-phenylpropionylamino)phenyl]-6H-thieno[3,2-f-][1,2,4]triazolo[4,3-a][1,4]diazepin-6-yl}acetate. In some embodiments, the thienotriazolodiazepine compound is (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide dihydrate.

In some embodiments, the thienotriazolodiazepine compound is formed as a solid dispersion comprising an amorphous thienotriazolodiazepine compound of the Formula (1) or a pharmaceutically acceptable salt thereof or a hydrate thereof; and a pharmaceutically acceptable polymer. In some embodiments, the solid dispersion exhibits an X-ray powder diffraction pattern substantially free of diffraction lines associated with crystalline thienotriazolodiazepine compound of Formula (1). In some embodiments, the solid dispersion exhibits a single glass transition temperature (Tg) inflection point ranging from about 130° C. to about 140° C. In some embodiments, the pharmaceutically acceptable polymer is hydroxypropylmethylcellulose acetate succinate having a thienotriazolodiazepine compound to hydroxypropylmethylcellulose acetate succinate (HPMCAS), weight ratio of 1:3 to 1:1.

In some embodiments, the patient has activated B-cell diffuse large B-cell lymphoma. In some embodiments, the activated B-cell diffuse large B-cell lymphoma has concomitant mutations in one or more of MYD88 gene, CD79B gene, CARD11 gene or wild type TP53 gene.

In some embodiments, the compound represented by Formula (1) down regulates expression of one or more genes of MYD88 gene, IRAK1 gene, TLR6 gene, IL6 gene, STAT3 gene, and TNFRSF17 gene. In some embodiments, the compound represented by Formula (1) down regulates expression of one or more genes involved in the NFKB pathway, said genes selected from IRF4, TNFAIP3 and BIRC3.

In some embodiments, the present invention provides methods of treating diffuse large B-cell lymphoma comprising administering to a patient a pharmaceutically acceptable amount of a composition comprising a thienotriazolodiazepine compound, said thienotriazolodiazepine compound being represented by the following Formula (1):

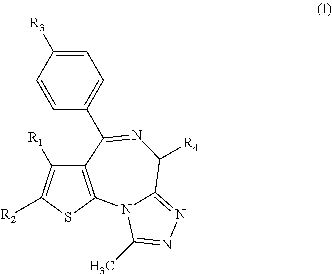

wherein R₁ is alkyl having a carbon number of 1-4, R₂ is a hydrogen atom; a halogen atom; or alkyl having a carbon number of 1-4 optionally substituted by a halogen atom or a hydroxyl group, R₃ is a halogen atom; phenyl optionally substituted by a halogen atom, alkyl having a carbon number of 1-4, alkoxy having a carbon number of 1-4 or cyano; —NR₅—(CH₂)ₘ—R₆ wherein R₅ is a hydrogen atom or alkyl having a carbon number of 1-4, m is an integer of 0-4, and R₆ is phenyl or pyridyl optionally substituted by a halogen atom; or —NR₇—CO—(CH₂)ₙ—R₈ wherein R₇ is a hydrogen atom or alkyl having a carbon number of 1-4, n is an integer of 0-2, and R₈ is phenyl or pyridyl optionally substituted by a halogen atom, and R₄ is —(CH₂)ₐ—CO—NH—R₉ wherein a is an integer of 1-4, and R₉ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR₁₀ wherein b is an integer of 1-4, and R₁₀ is alkyl having a carbon number of 1-4, or a pharmaceutically acceptable salt thereof or a hydrate or solvate thereof, wherein the thienotriazolodiazepine compound is formed as a solid dispersion comprising an amorphous thienotriazolodiazepine compound of the Formula (1) or a pharmaceutically acceptable salt thereof or a hydrate thereof, and a pharmaceutically acceptable polymer. In some embodiments, the patient has activated B-cell diffuse large B-cell lymphoma.

In some embodiments, the thienotriazolodiazepine compound represented by Formula 1 is independently selected from the group consisting of: (i) (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide or a dihydrate thereof, (ii) methyl (S)-{4-(3′-cyanobiphenyl-4-yl)-2,3,9-trimethyl-6H-thieno[3,2-f][1,2,4]tri-azolo[4,3-a][1,4]diazepin-6-yl}acetate, (iii) methyl (S)-{2,3,9-trimethyl-4-(4-phenylaminophenyl)-6H-thieno[3,2-f][1,2,4]triaz-olo[4,3-a][1,4]diazepin-6-yl}acetate; and (iv) methyl (S)-{2,3,9-trimethyl-4-[4-(3-phenylpropionylamino)phenyl]-6H-thieno[3,2-f-][1,2,4]triazolo[4,3-a][1,4]diazepin-6-yl}acetate. In some embodiments, the thienotriazolodiazepine compound is (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide dihydrate.

In some embodiments, the solid dispersion exhibits an X-ray powder diffraction pattern substantially free of diffraction lines associated with crystalline thienotriazolodiazepine compound of Formula (1). In some embodiments, the solid dispersion exhibits a single glass transition temperature (Tg) inflection point ranging from about 130° C. to about 140° C. In some embodiments, the pharmaceutically acceptable polymer is hydroxypropylmethylcellulose acetate succinate having a thienotriazolodiazepine compound to hydroxypropylmethylcellulose acetate succinate (HPMCAS), weight ratio of 1:3 to 1:1.

In some embodiments, the patient has activated B-cell diffuse large B-cell lymphoma. In some embodiments, the activated B-cell diffuse large B-cell lymphoma has concomitant mutations in one or more of MYD88 gene, CD79B gene, CARD11 gene or wild type TP53 gene.

In some embodiments, the compound represented by Formula (1) down regulates expression of one or more genes of MYD88 gene, IRAK1 gene, TLR6 gene, IL6 gene, STAT3 gene, and TNFRSF17 gene. In some embodiments, the compound represented by Formula (1) down regulates expression of one or more genes involved in the NFKB pathway, said genes selected from IRF4, TNFAIP3 and BIRC3.

### BRIEF DESCRIPTION OF THE DRAWINGS

The foregoing summary, as well as the following detailed description of embodiments of the methods of the present invention, will be better understood when read in conjunction with the appended drawings of exemplary embodiments. It should be understood, however, that the invention is not limited to the precise arrangements and instrumentalities shown.

In the drawings:

FIG. 1A illustrates dissolution profile of a comparator formulation comprising a solid dispersion comprising 25% compound (1-1) and Eudragit L100-55.

FIG. 1B illustrates dissolution profile of a comparator formulation comprising a solid dispersion comprising 50% compound (1-1) and Eudragit L100-55.

FIG. 1C illustrates dissolution profile of an exemplary formulation comprising a solid dispersion comprising 25% compound (1-1) and polyvinylpyrrolidone (PVP).

FIG. 1D illustrates dissolution profile of an exemplary formulation comprising a solid dispersion comprising 50% compound (1-1) and PVP.

FIG. 1E illustrates dissolution profile of an exemplary formulation comprising a solid dispersion comprising 25% compound (1-1) and PVP-vinyl acetate (PVP-VA).

FIG. 1F illustrates dissolution profile of an exemplary formulation comprising a solid dispersion comprising 50% compound (1-1) and PVP-VA.

FIG. 1G illustrates dissolution profile of an exemplary formulation comprising a solid dispersion comprising 25% compound (1-1) and hypromellose acetate succinate (HPMCAS-M).

FIG. 1H illustrates dissolution profile of an exemplary formulation comprising a solid dispersion comprising 50% compound (1-1) and HPMCAS-M.

FIG. 1I illustrates dissolution profile of an exemplary formulation comprising a solid dispersion comprising 25% compound (1-1) and hypromellose phthalate (HPMCP-HP55).

FIG. 1J illustrates dissolution profile of an exemplary formulation comprising a solid dispersion comprising 50% compound (1-1) and HMCP-HP55.

FIG. 2A illustrates results of in vivo screening of an exemplary formulation comprising a solid dispersion of 25% compound (1-1) and PVP.

FIG. 2B illustrates results of an in vivo screening of an exemplary formulation comprising a solid dispersion of 25% compound (1-1) and HPMCAS-M.

FIG. 2C illustrates results of an in vivo screening of an exemplary formulation comprising a solid dispersion of 50% compound (1-1) and HPMCAS-M.

FIG. 3 illustrates powder X-ray diffraction profiles of solid dispersions of compound (1-1).

FIG. 4A illustrates modified differential scanning calorimetry trace for a solid dispersion of 25% compound (1-1) and PVP equilibrated under ambient conditions.

FIG. 4B illustrates modified differential scanning calorimetry trace for a solid dispersion of 25% compound (1-1) and HPMCAS-M equilibrated under ambient conditions.

FIG. 4C illustrates modified differential scanning calorimetry trace for a solid dispersion of 50% compound (1-1) and HPMCAS-M equilibrated under ambient conditions.

FIG. 5 illustrates plot of glass transition temperature (Tg) versus relative hunidity (RH) for solid dispersions of 25% compound (1-1) and PVP or HMPCAS-M and 50% compound (1-1) and HPMCAS-MG.

FIG. 6 illustrates modified differential scanning calorimetry trace for a solid dispersion of 25% compound (1-1) and PVP equilibrated under 75% relative humidity.

FIGS. 7A and 7B illustrate plasma concentration versus time curves for Compound (1-1) after 1 mg/kg intravenous dosing (solid rectangles) and 3 mg/kg oral dosing as 25% Compound (1-1):PVP (open circles), 25% Compound (1-1):HPMCAS-MG (open triangles), and 50% Compound (1-1):HPMCAS-MG (open inverted triangles). The inset depicts the same data plotted on a semilogarithmic scale.

FIGS. 8A and 8B illustrate plasma concentration versus time curves for Compound (1-1) after 3 mg/kg oral dosing as 25% Compound (1-1): PVP (open circles), 25% Compound (1-1):HPMCAS-MG (open triangles), and 50% Compound (1-1):HPMCAS-MG (open inverted triangles). The inset depicts the same data plotted on a semi-logarithmic scale.

FIG. 9 illustrates a powder X-ray diffraction profile of solid dispersions of compound (1-1) in HPMCAS-MG at time zero of a stability test.

FIG. 10 illustrates a powder X-ray diffraction profile of solid dispersions of compound (1-1) in HPMCAS-MG after 1 month at 40° C. and 75% relative humidity.

FIG. 11 illustrates a powder X-ray diffraction profile of solid dispersions of compound (1-1) in HPMCAS-MG after 2 months at 40° C. and 75% relative humidity.

FIG. 12 illustrates a powder X-ray diffraction profile of solid dispersions of compound (1-1) in HPMCAS-MG after 3 month at 40° C. and 75% relative humidity.

FIG. 13 illustrates additive and synergistic effects of combinations of compound (1-1) with everolimus, lenalidomide, rituximab, decitabine, and vorinostat (Y-axis: confidence interval (CI)<0.3, strong synergism; 0.3-0.9, synergism; 0.9-1.1 additive effect) in germinal center B cell-like (GCB) cell lines (1: DOHH2; 2: Karpas422; 3: SUDHL6) and activated B cell-like (ABC) type of diffuse large B cell lymphoma (DLBCL) cell lines (4: U2932; and 5: TMD8).

### DETAILED DESCRIPTION OF THE INVENTION

The present subject matter will now be described more fully hereinafter with reference to the accompanying Figures and Examples, in which representative embodiments are shown. The present subject matter can, however, be embodied in different forms and should not be construed as limited to the embodiments set forth herein. Rather, these embodiments are provided to describe and enable one of skill in the art. Unless otherwise defined, all technical and scientific terms used herein have the same meaning as commonly understood by one of ordinary skill in the art to which the subject matter pertains. All publications, patent applications, patents, and other references mentioned herein are incorporated by reference in their entireties.

### I. DEFINITIONS

The term “alkyl group” as used herein refers to a saturated straight or branched hydrocarbon.

The term “substituted alkyl group” refers to an alkyl moiety having one or more substituents replacing a hydrogen or one or more carbons of the hydrocarbon backbone.

The term “alkenyl group” whether used alone or as part of a substituent group, for example, “C₁₋₄alkenyl(aryl),” refers to a partially unsaturated branched or straight chain monovalent hydrocarbon radical having at least one carbon-carbon double bond, whereby the double bond is derived by the removal of one hydrogen atom from each of two adjacent carbon atoms of a parent alkyl molecule and the radical is derived by the removal of one hydrogen atom from a single carbon atom. Atoms may be oriented about the double bond in either the cis (Z) or trans (E) conformation. Typical alkenyl radicals include, but are not limited to, ethenyl, propenyl, allyl(2-propenyl), butenyl and the like. Examples include C₁₋₄alkenyl or C₂₋₄alkenyl groups.

The term “C₍ⱼ₋ₖ₎” (where j and k are integers referring to a designated number of carbon atoms) refers to an alkyl, alkenyl, alkynyl, alkoxy or cycloalkyl radical or to the alkyl portion of a radical in which alkyl appears as the prefix root containing from j to k carbon atoms inclusive. For example, C₍₁₋₄₎ denotes a radical containing 1, 2, 3 or 4 carbon atoms.

The term “pharmaceutically acceptable salts” is art-recognized and refers to the relatively non-toxic, inorganic and organic acid addition salts, or inorganic or organic base addition salts of compounds, including, for example, those contained in compositions of the present invention.

The term “chiral” is art-recognized and refers to molecules That have the property of non-superimposability of the mirror image partner, while the term “achiral” refers to molecules which are superimposable on their mirror image partner. A “prochiral molecule” is a molecule that has the potential to be converted to a chiral molecule in a particular process.

The symbol “ [symbol] ” is used to denote a bond that may be a single, a double or a triple bond.

The term “enantiomer” as it used herein, and structural formulas depicting an enantiomer are meant to include the “pure” enantiomer free from its optical isomer as well as mixtures of the enantiomer and its optical isomer in which the enantiomer is present in an enantiomeric excess, e.g., at least 10%, 25%, 50%, 75%, 90%, 95%, 98%, or 99% enantiomeric excess.

The term “stereoisomers” when used herein consist of all geometric isomers, enantiomers or diastereomers. The present invention encompasses various stereoisomers of these compounds and mixtures thereof. Conformational isomers and rotamers of disclosed compounds are also contemplated.

The term “stereoselective synthesis” as it is used herein denotes a chemical or enzymatic reaction in which a single reactant forms an unequal mixture of stereoisomers during the creation of a new stereocenter or during the transformation of a pre-existing one, and are well known in the art. Stereoselective syntheses encompass both enantioselective and diastereoselective transformations. For examples, see Carreira, E. M. and Kvaerno, L., Classics in Stereoselective Synthesis, Wiley-VCH: Weinheim, 2009.

The term “pharmaceutically acceptable salts” is art-recognized and refers to the relatively non-toxic, inorganic and organic acid addition salts, or inorganic or organic base addition salts of compounds, including, for example, those contained in compositions of the present invention.

The term “spray drying” refers to processes which involve the atomization of the feed suspension or solution into small droplets and rapidly removing solvent from the mixture in a processor chamber where there is a strong driving force for the evaporation (i.e., hot dry gas or partial vacuum or combinations thereof).

As used herein, the term “effective amount” refers to an amount of a hienopyrazolodiazapine of the present invention or any other pharmaceutically active agent that will elicit a targeted biological or a medical response of a tissue, a biological system, an animal or a human, for instance, intended by a researcher or clinician or a healthcare provider. In some embodiments, the term “effective amount” is used to refer any amount of a thienotriazolodiazapine of the present invention or any other pharmaceutically active agent which is effective at enhancing a normal physiological function.

The term “therapeutically effective amount” as used herein refers to any amount of a thienotriazolodiazapine of the present invention or any other pharmaceutically active agent which, as compared to a corresponding a patient who has not received such an amount of the thienotriazolodiazapine or the other pharmaceutically active agent, results in improved treatment, healing, prevention, or amelioration of a disease, disorder, or side effect, or a decrease in the rate of advancement of a disease or disorder.

Throughout this application and in the claims that follow, unless the context requires otherwise, the word “comprise”, or variations such as “comprises” or “comprising”, should be understood to imply the inclusion of a stated integer step or group of integers or steps but not the exclusion of any other integer or step or group of integers or steps.

### II. METHOD OF USE

The present inventions described herein provide for methods of treating lymphoma. The detailed description sets forth the disclosure in various parts: III. Thienotriazolodiazepine Compounds; IV. Formulations; V. Dosage Forms; VI. Dosage; VII. Process; and VIII. Examples. One of skill in the art would understand that each of the various embodiments of methods of treatment include the various embodiments of thienotriazolodiazepine compounds, formulations, dosage forms, dosage and processes described herein.

In some embodiments, the present invention provides methods of treating diffuse large B-cell lymphoma comprising administering to a patient a pharmaceutically acceptable amount of a composition comprising a thienotriazolodiazepine compound, said thienotriazolodiazepine compound being represented by the following Formula (1):

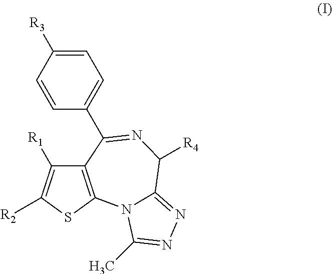

wherein R₁ is alkyl having a carbon number of 1-4, R₂ is a hydrogen atom; a halogen atom; or alkyl having a carbon number of 1-4 optionally substituted by a halogen atom or a hydroxyl group, R₃ is a halogen atom; phenyl optionally substituted by a halogen atom, alkyl having a carbon number of 1-4, alkoxy having a carbon number of 1-4 or cyano; —NR₅—(CH₂)ₘ—R₆ wherein R₅ is a hydrogen atom or alkyl having a carbon number of 1-4, m is an integer of 0-4, and R₆ is phenyl or pyridyl optionally substituted by a halogen atom; or —NR₇—CO—(CH₂)ₙ—R₈ wherein R₇ is a hydrogen atom or alkyl having a carbon number of 1-4, n is an integer of 0-2, and R₈ is phenyl or pyridyl optionally substituted by a halogen atom, and R₄ is —(CH₂)ₐ—CO—NH—R₉ wherein a is an integer of 1-4, and R₉ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR₁₀ wherein b is an integer of 1-4, and R₁₀ is alkyl having a carbon number of 1-4, or a pharmaceutically acceptable salt thereof or a hydrate or solvate thereof. In some embodiments, the patient has activated B-cell diffuse large B-cell lymphoma.

In some embodiments, the thienotriazolodiazepine compound represented by Formula 1 is independently selected from the group consisting of: (i) (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide or a dihydrate thereof, (ii) methyl (S)-{4-(3′-cyanobiphenyl-4-yl)-2,3,9-trimethyl-6H-thieno[3,2-f][1,2,4]tri-azolo[4,3-a][1,4]diazepin-6-yl}acetate, (iii) methyl (S)-{2,3,9-trimethyl-4-(4-phenylaminophenyl)-6H-thieno[3,2-f][1,2,4]triaz-olo[4,3-a][1,4]diazepin-6-yl}acetate; and (iv) methyl (S)-{2,3,9-trimethyl-4-[4-(3-phenylpropionylamino)phenyl]-6H-thieno[3,2-f-][1,2,4]triazolo[4,3-a][1,4]diazepin-6-yl}acetate. In some embodiments, the thienotriazolodiazepine compound is (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide dihydrate.

In some embodiments, the thienotriazolodiazepine compound of Formula (1) is formed as a solid dispersion comprising an amorphous thienotriazolodiazepine compound of Formula (1) and a pharmaceutically acceptable salt thereof or a hydrate thereof; and a pharmaceutically acceptable polymer. Various embodiments of such a solid dispersion are described herein and can be used accordingly.

In some embodiments, the solid dispersion exhibits an X-ray powder diffraction pattern substantially free of diffraction lines associated with crystalline thienotriazolodiazepine compound of Formula (1). In some embodiments, the solid dispersion exhibits a single glass transition temperature (Tg) inflection point ranging from about 130° C. to about 140° C. In some embodiments, the pharmaceutically acceptable polymer is hydroxypropylmethylcellulose acetate succinate having a thienotriazolodiazepine compound to hydroxypropylmethylcellulose acetate succinate (HPMCAS), weight ratio of 1:3 to 1:1.

In some embodiments, the patient has activated B-cell diffuse large B-cell lymphoma. In some embodiments, the activated B-cell diffuse large B-cell lymphoma has concomitant mutations in one or more of MYD88 gene, CD79B gene, CARD11 gene or wild type TP53 gene.

In some embodiments, the compound represented by Formula (1) down regulates expression of one or more genes of MYD88 gene, IRAK1 gene, TLR6 gene, IL6 gene, STAT3 gene, and TNFRSF17 gene. In some embodiments, the compound represented by Formula (1) down regulates expression of one or more genes involved in the NFKB pathway, said genes selected from IRF4, TNFAIP3 and BIRC3.

In one embodiment of the methods of treating a cancer in a mammal, the gene expression profile of the mammal's cancer cells is negative for one or more of BCL2L1/BCLX1, BIRC5/survivin, ERCC1, TAF1A and BRD7.

Suitable mammalian target of rapamycin (mTOR) inhibitors for use in combinations with the thienotriazolodiazapine of Formula (1) in the methods of the present invention include, but are not limited to, the mTOR inhibitors listed in the below Table A.

In some embodiments, a second compound selected from the group consisting of mTOR inhibitor and BTK inhibitor is administered in combination with the thienotriazolodiazepine of Formula (1). In some embodiments the thienotrazolodiazepine and the second compound can be administered simultaneously, while in other embodiments the thienotriazolodiazepine compound and the second compound can be administered sequentially. In some embodiments the combination produces a synergistic effect.

In some embodiments, the present invention provides methods of treating diffuse large B-cell lymphoma comprising administering to a patient a pharmaceutically acceptable amount of a composition comprising a thienotriazolodiazepine compound, said thienotriazolodiazepine compound being represented by the following Formula (1):

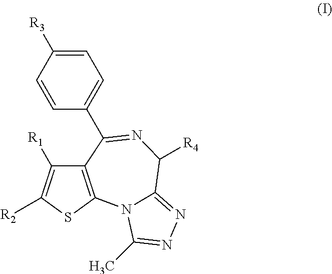

wherein R₁ is alkyl having a carbon number of 1-4, R₂ is a hydrogen atom; a halogen atom; or alkyl having a carbon number of 1-4 optionally substituted by a halogen atom or a hydroxyl group, R₃ is a halogen atom; phenyl optionally substituted by a halogen atom, alkyl having a carbon number of 1-4, alkoxy having a carbon number of 1-4 or cyano; —NR₅—(CH₂)ₘ—R₆ wherein R₅ is a hydrogen atom or alkyl having a carbon number of 1-4, m is an integer of 0-4, and R₆ is phenyl or pyridyl optionally substituted by a halogen atom; or —NR₇—CO—(CH₂)ₙ—R₈ wherein R₇ is a hydrogen atom or alkyl having a carbon number of 1-4, n is an integer of 0-2, and R₈ is phenyl or pyridyl optionally substituted by a halogen atom, and R₄ is —(CH₂)ₐ—CO—NH—R₉ wherein a is an integer of 1-4, and R₉ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR₁₀ wherein b is an integer of 1-4, and R₁₀ is alkyl having a carbon number of 1-4, or a pharmaceutically acceptable salt thereof or a hydrate or solvate thereof, wherein the thienotriazolodiazepine compound of Formula (1) is formed as a solid dispersion comprising an amorphous thienotriazolodiazepine compound of Formula (1) and a pharmaceutically acceptable salt thereof or a hydrate thereof; and a pharmaceutically acceptable polymer. Various embodiments of such a solid dispersion are described herein and can be used accordingly. In some embodiments, the patient has activated B-cell diffuse large B-cell lymphoma.

In some embodiments, the thienotriazolodiazepine compound represented by Formula 1 is independently selected from the group consisting of: (i) (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide or a dihydrate thereof, (ii) methyl (S)-{4-(3′-cyanobiphenyl-4-yl)-2,3,9-trimethyl-6H-thieno[3,2-f][1,2,4]tri-azolo[4,3-a][1,4]diazepin-6-yl}acetate, (iii) methyl (S)-{2,3,9-trimethyl-4-(4-phenylaminophenyl)-6H-thieno[3,2-f][1,2,4]triaz-olo[4,3-a][1,4]diazepin-6-yl}acetate; and (iv) methyl (S)-{2,3,9-trimethyl-4-[4-(3-phenylpropionylamino)phenyl]-6H-thieno[3,2-f-][1,2,4]triazolo[4,3-a][1,4]diazepin-6-yl}acetate. In some embodiments, the thienotriazolodiazepine compound is (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide dihydrate.

In some embodiments, the solid dispersion exhibits an X-ray powder diffraction pattern substantially free of diffraction lines associated with crystalline thienotriazolodiazepine compound of Formula (1). In some embodiments, the solid dispersion exhibits a single glass transition temperature (Tg) inflection point ranging from about 130° C. to about 140° C. In some embodiments, the pharmaceutically acceptable polymer is hydroxypropylmethylcellulose acetate succinate having a thienotriazolodiazepine compound to hydroxypropylmethylcellulose acetate succinate (HPMCAS), weight ratio of 1:3 to 1:1.

In some embodiments, the patient has activated B-cell diffuse large B-cell lymphoma. In some embodiments, the activated B-cell diffuse large B-cell lymphoma has concomitant mutations in one or more of MYD88 gene, CD79B gene, CARD11 gene or wild type TP53 gene.

In some embodiments, the compound represented by Formula (1) down regulates expression of one or more genes of MYD88 gene, IRAK1 gene, TLR6 gene, IL6 gene, STAT3 gene, and TNFRSF17 gene. In some embodiments, the compound represented by Formula (1) down regulates expression of one or more genes involved in the NFKB pathway, said genes selected from IRF4, TNFAIP3 and BIRC3.

In one embodiment of the methods of treating a cancer in a mammal, the gene expression profile of the mammal's cancer cells is negative for one or more of BCL2L1/BCLX1, BIRC5/survivin, ERCC1, TAF1A and BRD7.

Suitable mammalian target of rapamycin (mTOR) inhibitors for use in combinations with the thienotriazolodiazapine of Formula (1) in the methods of the present invention include, but are not limited to, the mTOR inhibitors listed in the below Table A.

In some embodiments, a second compound selected from the group consisting of mTOR inhibitor and BTK inhibitor is administered in combination with the thienotriazolodiazepine of Formula (1). In some embodiments the thienotrazolodiazepine and the second compound can be administered simultaneously, while in other embodiments the thienotriazolodiazepine compound and the second compound can be administered sequentially. In some embodiments the combination produces a synergistic effect.

A mammalian subject as used herein can be any mammal. In one embodiment, the mammalian subject includes, but is not limited to, a human; a non-human primate; a rodent such as a mouse, rat, or guinea pig; a domesticated pet such as a cat or dog; a horse, cow, pig, sheep, goat, or rabbit. In one embodiment, the mammalian subject includes, but is not limited to, a bird such as a duck, goose, chicken, or turkey. In one embodiment, the mammalian subject is a human. In one embodiment, the mammalian subject can be either gender and can be any age.

TABLE A

| No. | Inhibitor Name | Description | Literature Citations |
|---|---|---|---|
| 1 | BEZ235 (NVP-BEZ235) 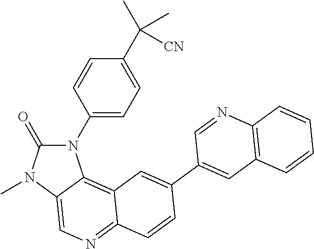 [SMILES: Cn1c(=O)n(-c2ccc(C(C)(C)C#N)cc2)c2c3cc(-c4cnc5ccccc5c4)ccc3ncc21] | BEZ235 (NVP- BEZ235) is a dual ATP- competitive PI3K and mTOR inhibitor of p110α, p110γ, p110δ and p110β with IC50 of 4 nM, 5 nM, 7 nM and 75 nM, respectively, and also inhibits ATR with IC50 of 21 nM. | Nature, 2012, 487(7408):505- 9; Blood, 2011, 118(14), 3911- 3921;Cancer Res, 2011, 71(15), 5067-5074. |
| 2 | Everolimus (RAD001) 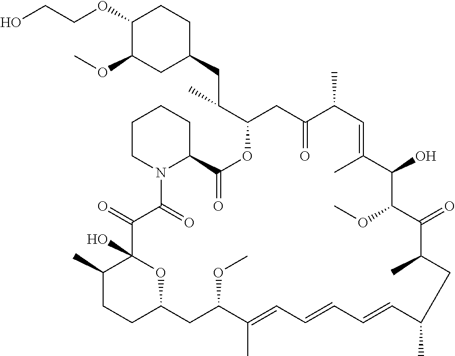 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] | Everolimus (RAD001) is an mTOR inhibitor of FKBP12 with IC50 of 1.6-2.4 nM. | Cell, 2012, 149(3):656- 70;;Cancer Cell, 2012, 21(2), 155- 167; Clin Cancer Res, 2013, 19(3):598-609. |
| 3 | Rapamycin (Sirolimus, AY22989, NSC226080) 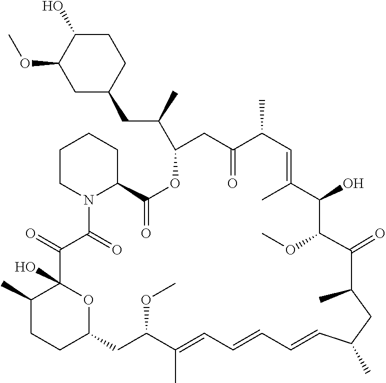 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C] | Rapamycin (Sirolimus, AY-22989, WY- 090217) is a specific mTOR inhibitor with IC50 of ~0.1 nM. | Cancer Cell, 2011, 19(6), 792- 804;;Cancer Res, 2013, ;Cell Res, 2012, 22(6):1003-21. |
| 4 | AZD8055 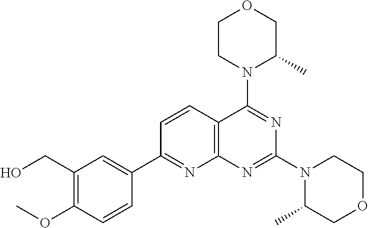 [SMILES: COc1ccc(-c2ccc3c(N4CCOC[C@@H]4C)nc(N4CCOC[C@@H]4C)nc3n2)cc1CO] | AZD8055 is a novel ATP-competitive inhibitor of mTOR with IC50 of 0.8 nM. | Autophagy, 2012, Am J Transplant, 2013, ;Biochem Pharmacol, 2012, 83(9), 1183-1194 |
| 5 | PI-103 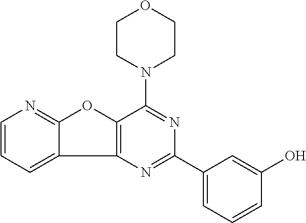 [SMILES: Oc1cccc(-c2nc(N3CCOCC3)c3oc4ncccc4c3n2)c1] 3-[4-(4-Morpholinylpyrido[3′,2′:4,5]f-uro[3,2-d]pyrimidin-2-yl]phenol | PI-103 is a potent, ATP- competitive PI3K inhibitor of DNA-PK, p110α, mTORC1, PI3KC2β, p110δ, mTORC2, p110β, and p110γ with IC50 of 2 nM, 8 nM, 20 nM, 26 nM, 48 nM, 83 nM, 88 nM and 150 nM, respectively. | Leukemia, 2013, 27(3):650- 60;Leukemia, 2012, 26(5):927- 33;Biochem Pharmacol, 2012, 83(9), 1183-1194. |
| 6 | Temsirolimus (CCI-779, NSC-683864) 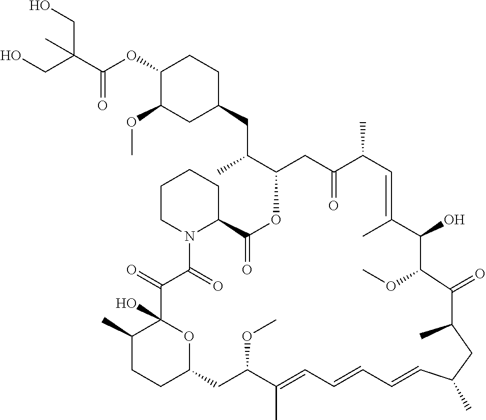 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C] | Temsirolimus (CCI-779, Torisel) is a specific mTOR inhibitor with IC50 of 1.76 μM. | Autophagy, 2011, 7(2), 176- 187;Tuberc Respir Dis (Seoul), 2012, 72(4), 343-351;PLoS One, 2013, 8(5):e62104. |
| 7 | Ku-0063794 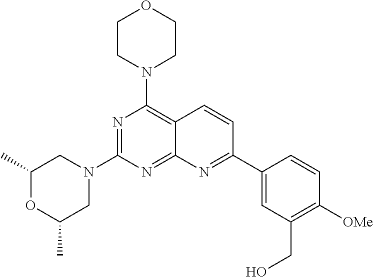 [SMILES: COc1ccc(-c2ccc3c(N4CCOCC4)nc(N4C[C@@H](C)O[C@@H](C)C4)nc3n2)cc1CO] rel-5-[2-[(2R,6S)-2,6-dimethyl-4-mo-rpholinyl]-4-(4- morpholinyl)pyrido[2,3-d]pyrimidin- 7-yl]-2-methoxybenzenemethanol | KU-0063794 is a potent and highly specific mTOR inhibitor for both mTORC1 and mTORC2 with IC50 ~10 nM. | Cell Stem Cell, 2012, 10(2):210- 7;Circ Res, 2010, 107(10), 1265- 1274;J Immunol, 2013, 190(7), 3246-55. |
| 8 | GDC-0349 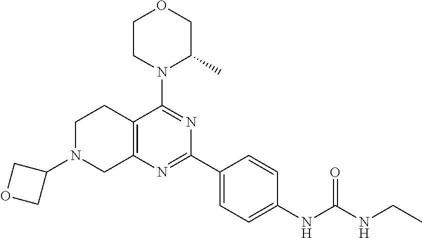 [SMILES: CCNC(=O)Nc1ccc(-c2nc3c(c(N4CCOC[C@@H]4C)n2)CCN(C2COC2)C3)cc1] | GDC-0349, is a potent and selective ATP- competitive inhibitor of mTOR with Ki of 3.8 nM. | |
| 9 | Torin 2 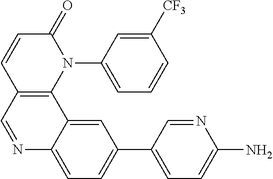 [SMILES: Nc1ccc(-c2ccc3ncc4ccc(=O)n(-c5cccc(C(F)(F)F)c5)c4c3c2)cn1] 9-(6-Amino-3-pyridinyl)-1-[3-(trifl-uoromethyl)pheny1]- benzo[h]-1,6-naphthyridin-2(1H)-one | Torin 2 is a highly potent and selective mTOR inhibitor with IC50 of 0.25 nM, and also exhibits potent cellular activity against ATM/ATR/DNA-PK with EC50 of 28 nM, 35 nM and 118 nM, respectively. | |
| 10 | INK 128 (MLN-0128) 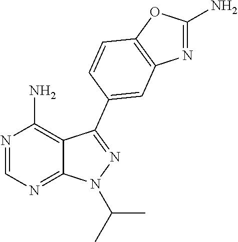 [SMILES: CC(C)n1nc(-c2ccc3oc(N)nc3c2)c2c(N)ncnc21] | INK 128 is a potent and selective mTOR inhibitor with IC50 of 1 nM. | |
| 11 | AZD2014 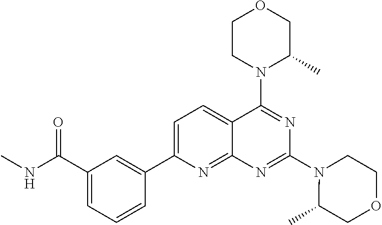 [SMILES: CNC(=O)c1cccc(-c2ccc3c(N4CCOC[C@@H]4C)nc(N4CCOC[C@@H]4C)nc3n2)c1] | AZD2014 is a novel dual mTORC1 and mTORC2 inhibitor with potential antineoplastic activity. | |
| 12 | NVP-BGT226(BGT226) 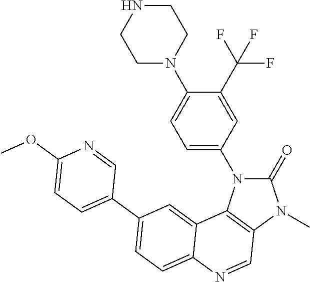 | NVP-BGT226 is a novel dual PI3K/mTOR inhibitor with IC50 of 1 nM. | |
| 13 | PF-04691502 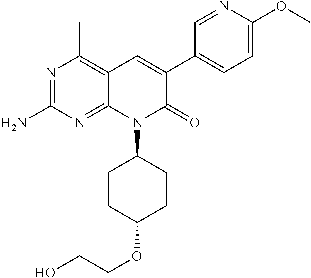 [SMILES: COc1ccc(-c2cc3c(C)nc(N)nc3n([C@H]3CC[C@H](OCCO)CC3)c2=O)cn1] | PF-04691502 is an ATP-competitive, selective inhibitor of PI3K(α/β/δ/γ)/mTOR with Ki of 1.8 nM/2.1 nM/1.6 nM/1.9 nM and 16 nM, also inhibits Akt phosphorylation on T308/S473 with IC50 of 7.5 nM/3.8 nM. | |
| 14 | CH5132799 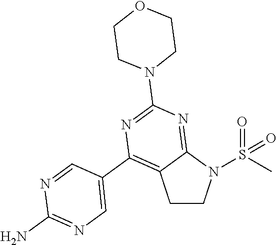 [SMILES: CS(=O)(=O)N1CCc2c(-c3cnc(N)nc3)nc(N3CCOCC3)nc21] | CH5132799 exhibits a strong inhibitory activity especially against PI3Kα with IC50 of 14 nM and also inhibits mTOR with IC50 of 1.6 μM. | |
| 15 | GDC-0980 (RG7422) 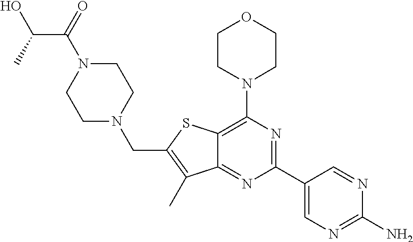 [SMILES: Cc1c(CN2CCN(C(=O)[C@H](C)O)CC2)sc2c(N3CCOCC3)nc(-c3cnc(N)nc3)nc12] | GDC-0980 (RG7422) is a potent, selective inhibitor of PI3Kα, PI3Kβ, PI3Kδ and PI3Kγ with IC50 of 5 nM, 27 nM, 7 nM, and 14 nM, and also a mTOR inhibitor with Ki of 17 nM. | |
| 16 | Torin 1 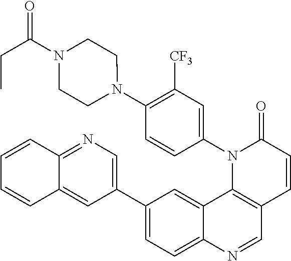 [SMILES: CCC(=O)N1CCN(c2ccc(-n3c(=O)ccc4cnc5ccc(-c6cnc7ccccc7c6)cc5c43)cc2C(F)(F)F)CC1] 1-[4-[4-(1-Oxopropyl)-1-piperazinyl-]-3-(trifluoromethyl)phenyl]- 9-(3-quinolinyl)-benz-o[h]-1,6-naphthyridin-2(1H)-one | Torin 1 is a potent inhibitor of mTOR with IC50 of 2-10 nM. | |
| 17 | WAY-600 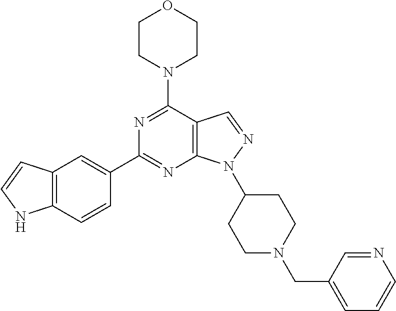 [SMILES: c1cncc(CN2CCC(n3ncc4c(N5CCOCC5)nc(-c5ccc6[nH]ccc6c5)nc43)CC2)c1] | WAY-600 is a potent, ATP-competitive and selective inhibitor of mTOR with 1050 of 9 nM. | |
| 18 | WYE-125132(WYE-132) 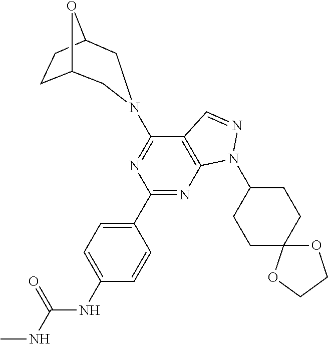 [SMILES: CNC(=O)Nc1ccc(-c2nc(N3CC4CCC(C3)O4)c3cnn(C4CCC5(CC4)OCCO5)c3n2)cc1] | WYE-125132 is a highly potent, ATP- competitive and specific mTOR inhibitor with IC50 of 0.19 nM. | |
| 19 | WYE-687 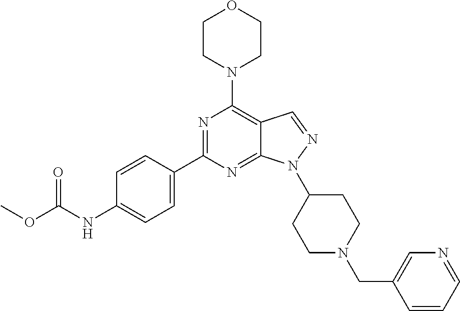 [SMILES: COC(=O)Nc1ccc(-c2nc(N3CCOCC3)c3cnn(C4CCN(Cc5cccnc5)CC4)c3n2)cc1] | WYE-687 is an ATP- competitive and selective inhibitor of mTOR with IC50 of 7 nM. | |
| 20 | GSK2126458(GSK458) 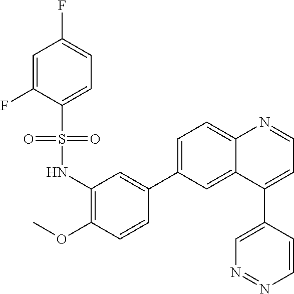 [SMILES: COc1ccc(-c2ccc3nccc(-c4ccnnc4)c3c2)cc1NS(=O)(=O)c1ccc(F)cc1F] | GSK2126458 is a highly selective and potent inhibitor of p110α, p110β, p110γ,p110δ, mTORC1 and mTORC2 with Ki of 0.019 nM, 0.13 nM, 0.024 nM, 0.06 nM, 0.18 nM and 0.3 nM, respectively. | |
| 21 | PF-05212384 (PKI-587) 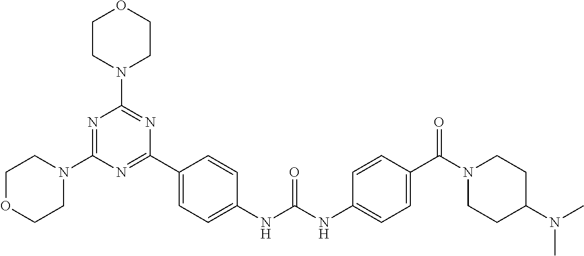 [SMILES: CN(C)C1CCN(C(=O)c2ccc(NC(=O)Nc3ccc(-c4nc(N5CCOCC5)nc(N5CCOCC5)n4)cc3)cc2)CC1] | PKI-587 is a highly potent dual inhibitor of PI3Kα, PI3Kγ and mTOR with IC50 of 0.4 nM, 5.4 nM and 1.6 nM, respectively. | |
| 22 | PP-121 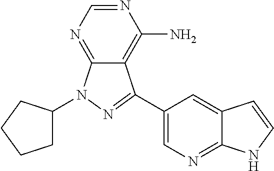 [SMILES: Nc1ncnc2c1c(-c1cnc3[nH]ccc3c1)nn2C1CCCC1] 1-Cyclopentyl-3-(1H-pyrrolo[2,3-b]p-yridin- 5-yl)-1H-pyrazolo[3,4-d]pyrimidin-4-amine | PP-121 is a multi-target inhibitor of PDGFR, Hck, mTOR, VEGFR2, Src and Abl with IC50 of 2 nM, 8 nM, 10 nM, 12 nM, 14 nM and 18 nM, respectively, and also inhibits DNA-PK with IC50 of 60 nM. | |
| 23 | OSI-027(ASP4786) 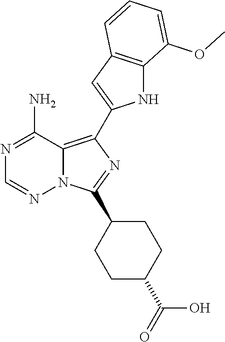 [SMILES: COc1cccc2cc(-c3nc([C@H]4CC[C@H](C(=O)O)CC4)n4ncnc(N)c34)[nH]c12] | OSI-027 is a selective and potent dual inhibitor of mTORC1 and mTORC2 with IC50 of 22 nM and 65 nM, respectively. | Exp Eye Res, 2013, 113C, 9-18 |
| 24 | Palomid 529(P529) 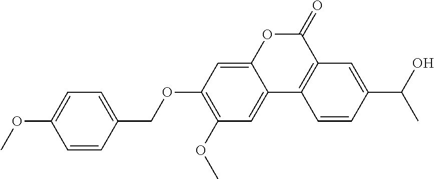 [SMILES: COc1ccc(COc2cc3oc(=O)c4cc(C(C)O)ccc4c3cc2OC)cc1] | Palomid 529 inhibits both the mTORC1 and mTORC2 complexes, reduces phosphorylation of pAktS473, pGSK3βS9, and pS6 but neither pMAPK nor pAktT308. Phase 1. | |
| 25 | PP242 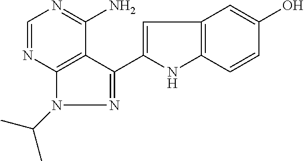 [SMILES: CC(C)n1nc(-c2cc3cc(O)ccc3[nH]2)c2c(N)ncnc21] 2-[4-Amino-1-(1-methylethyl)-1H-pyr-azolo[3,4- d]pyrimidin-3-yl]-1H-indol-5-ol | PP242 is a selective mTOR inhibitor with IC50 of 8 nM. | Autophagy, 2012, 8(6), 903-914 |
| 26 | XL765(SAR245409) 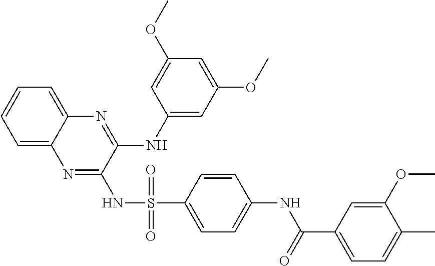 [SMILES: COc1cc(Nc2nc3ccccc3nc2NS(=O)(=O)c2ccc(NC(=O)c3ccc(C)c(OC)c3)cc2)cc(OC)c1] | XL765 is a dual inhibitor of mTOR/PI3k for mTOR, p110α, p110β, p110γ and p110δ with IC50 of 157 nM, 39 nM, 113 nM, 9 nM and 43 nM, respectively. | Endocrinology, 2013, 154(3):1247-59 |
| 27 | GSK1059615 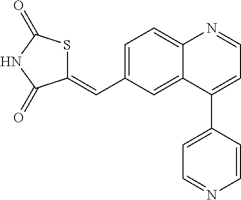 [SMILES: O=C1NC(=O)/C(=C/c2ccc3nccc(-c4ccncc4)c3c2)S1] 5-[[4-(4-Pyridinyl)-6-quinolinyl]me-thylene]-2,4-thiazolidenedione | GSK1059615 is a novel and dual inhibitor of PI3Kα, P13Kβ, PI3Kδ, PI3Kγ and mTOR with IC50 of 0.4 nM, 0.6 nM, 2 nM, 5 nM and 12 nM, respectively. | Nature, 2012, 486(7404), 532-536 |
| 28 | WYE-354 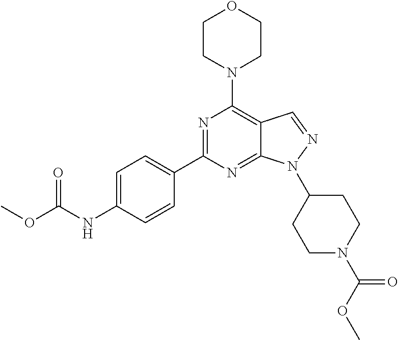 [SMILES: COC(=O)Nc1ccc(-c2nc(N3CCOCC3)c3cnn(C4CCN(C(=O)OC)CC4)c3n2)cc1] | WYE-354 is a potent, specific and ATP- competitive inhibitor of mTOR with 1050 of 5 nM. | Mol Cancer Res, 2012, 10(6), 821-833. |
| 29 | Deforolimus (Ridaforolimus, MK-8669) 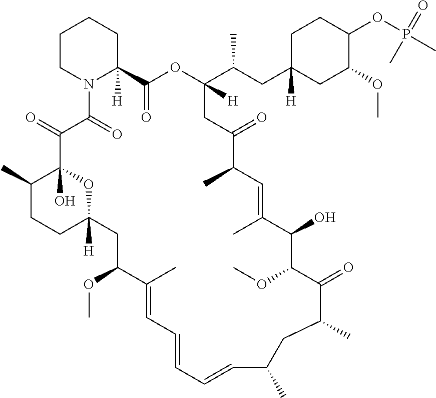 | Deforolimus (Ridaforolimus; AP23573; MK-8669; 42-(Dimethylphosphinate) rapamycin; Ridaforolimus) is a selective mTOR inhibitor with 1050 of 0.2 nM. | Mol Genet Meta, 2010, 100(4), 309-315. |

Suitable Bruton's tyrosine kinase (BKT) inhibitors for use in combinations with the thienopyrazolodiazapine of Formula (1) in the methods of the present invention include, but are not limited to, the BKT inhibitors listed in the below Table B.

TABLE B

| Inhibitor Name | Inhibitor Information | Literature Citations |
|---|---|---|
| PCI-32765 (Ibrutinib) 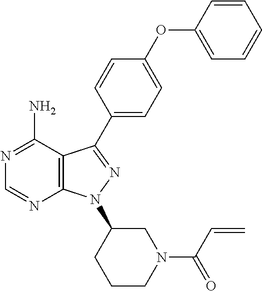 [SMILES: C=CC(=O)N1CCC[C@@H](n2nc(-c3ccc(Oc4ccccc4)cc3)c3c(N)ncnc32)C1] | PCI-32765 (Ibrutinib) is a potent and highly selective Btk inhibitor with IC50 of 0.5 nM. | Cancer Cell, 2012, 22(5):656-67. Blood, 2012, 120(19), 3978-3985; Cell Signal, 2013, 25(1):106-12. |
| GDC-0834 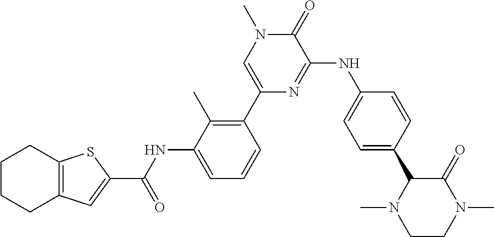 | GDC-0834 is a novel potent and selective BTK inhibitor with IC50 of 5.9 nM. | |
| LFM-A13 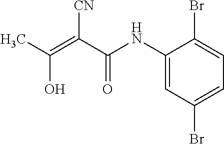 [SMILES: C/C(O)=C(\C#N)C(=O)Nc1cc(Br)ccc1Br] | Bruton's tyrosine kinase (BTK) inhibitor IC50 = 2.5 μM. IC50's for JAK- 1, JAK-2, JAK-3, SYK, HCK, EGFR kinase, IR kinase all >300 μM | |
| Terreic acid 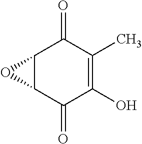 (1R,6S)-3-Hydroxy-4-methyl-7- oxabicyclo[4.1.0]hept-3-ene-2,5-dione | Selective inhibitor of Bruton's tyrosine kinase (BTK). Inhibits the catalytic activity of BTK as well as the interaction between BTK and PKCβII, in intact cells and in cell-free systems, without affecting the activity of PKC. Terreic acid has little or no effect on the activities of Lyn, Syk, PKA, casein kinase I, ERK1, ERK2 and p38 kinase. A useful tool in studying the role of BTK in cellular signaling. | |

### III. THIENOTRIAZOLODIAZEPINE COMPOUNDS

In one embodiment, the thienotriazolodiazepine compounds, used in the formulations of the present invention, are represented by Formula (1):

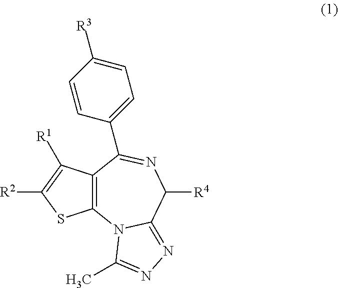

wherein R¹ is alkyl having a carbon number of 1-4, R² is a hydrogen atom; a halogen atom; or alkyl having a carbon number of 1-4 optionally substituted by a halogen atom or a hydroxyl group, R³ is a halogen atom; phenyl optionally substituted by a halogen atom, alkyl having a carbon number of 1-4, alkoxy having a carbon number of 1-4 or cyano; —NR⁵—(CH₂)ₘ—R⁶ wherein R⁵ is a hydrogen atom or alkyl having a carbon number of 1-4, m is an integer of 0-4, and R⁶ is phenyl or pyridyl optionally substituted by a halogen atom; or —NR⁷—CO—(CH₂)ₙ—R⁸ wherein R⁷ is a hydrogen atom or alkyl having a carbon number of 1-4, n is an integer of 0-2, and R⁸ is phenyl or pyridyl optionally substituted by a halogen atom, and R⁴ is —(CH₂)ₐ—CO—NH—R⁹ wherein a is an integer of 1-4, and R⁹ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR¹⁰ wherein b is an integer of 1-4, and R¹⁰ is alkyl having a carbon number of 1-4, including any salts, isomers, enantiomers, racemates, hydrates, solvates, metabolites, and polymorphs thereof.

In one embodiment, a suitable alkyl group includes linear or branched akyl radicals including from 1 carbon atom up to 4 carbon atoms. In one embodiment, a suitable alkyl group includes linear or branched akyl radicals including from 1 carbon atom up to 3 carbon atoms. In one embodiment, a suitable alkyl group includes linear or branched akyl radicals include from 1 carbon atom up to 2 carbon atoms. In one embodiment, exemplary alkyl radicals include, but are not limited to, methyl, ethyl, propyl, isopropyl, n-butyl, isobutyl, sec-butyl, tert-butyl. In one embodiment, exemplary alkyl groups include, but are not limited to, methyl, ethyl, propyl, isopropyl, 2-methyl-1-propyl, and 2-methyl-2-propyl.

In some embodiments, the present invention provides pharmaceutically acceptable salts, solvates, including hydrates, and isotopically-labeled forms of the thienotriazolodiazepine compounds described herein. In one embodiment, pharmaceutically acceptable salts of the thienotriazolodiazepine compounds include acid addition salts formed with inorganic acids. In one embodiment, pharmaceutically acceptable, inorganic acid addition salts of the thienotriazolodiazepine include salts of hydrochloric, hydrobromic, hydroiodic, phosphoric, metaphosphoric, nitric and sulfuric acids. In one embodiment, pharmaceutically acceptable salts of the thienotriazolodiazepine compounds include acid addition salts formed with organic acids. In one embodiment, pharmaceutically acceptable organic acid addition salts of the thienotriazolodiazepine include salts of tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and 4-methyl benzenesulfonic acids.

Representative thienotriazolodiazepine compounds of Formula (1) include, but are not limited to, the thienotriazolodiazepine compounds (1-1) to (1-18), which are listed in the following Table C.

Compound (1-1), of Table C, will be referred to herein as OTX-015 or Y-803.

TABLE C
Exemplary compounds of the invention:

| | |
|---|---|
| 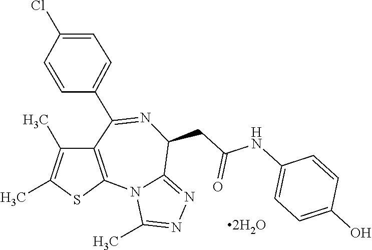 [SMILES: Cc1sc2c(c1C)C(c1ccc(Cl)cc1)=N[C@@H](CC(=O)Nc1ccc(O)cc1)c1nnc(C)n1-2] | (1-1) |
| 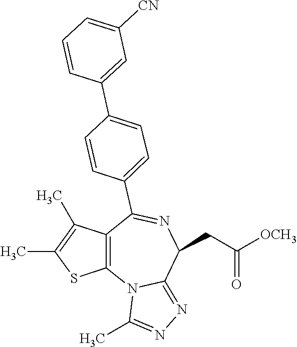 [SMILES: COC(=O)C[C@@H]1N=C(c2ccc(-c3cccc(C#N)c3)cc2)c2c(sc(C)c2C)-n2c(C)nnc21] | (1-2) |
| 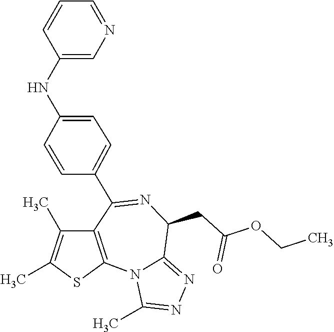 [SMILES: CCOC(=O)C[C@@H]1N=C(c2ccc(Nc3cccnc3)cc2)c2c(sc(C)c2C)-n2c(C)nnc21] | (1-3) |
| 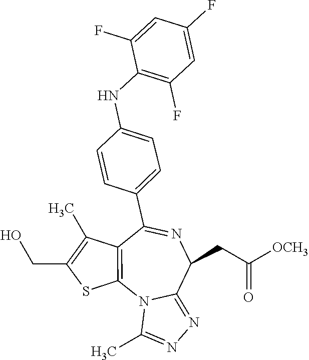 [SMILES: COC(=O)C[C@@H]1N=C(c2ccc(Nc3c(F)cc(F)cc3F)cc2)c2c(sc(CO)c2C)-n2c(C)nnc21] | (1-4) |
| 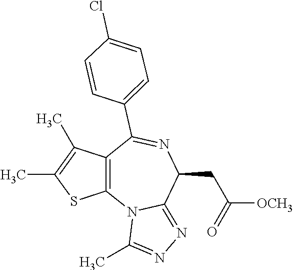 [SMILES: COC(=O)C[C@@H]1N=C(c2ccc(Cl)cc2)c2c(sc(C)c2C)-n2c(C)nnc21] | (1-5) |
| 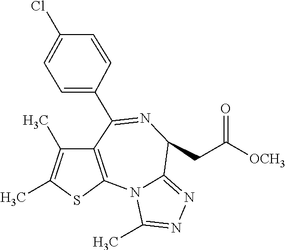 [SMILES: COC(=O)C[C@@H]1N=C(c2ccc(Cl)cc2)c2c(sc(C)c2C)-n2c(C)nnc21] | (1-6) |
| 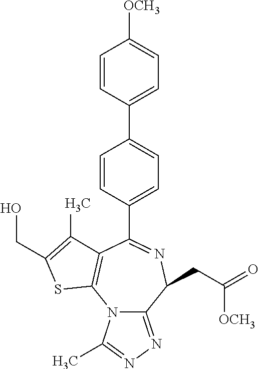 [SMILES: COC(=O)C[C@@H]1N=C(c2ccc(-c3ccc(OC)cc3)cc2)c2c(sc(CO)c2C)-n2c(C)nnc21] | (1-7) |
| 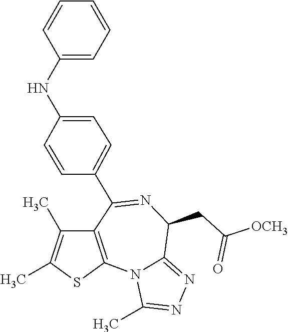 [SMILES: COC(=O)C[C@@H]1N=C(c2ccc(Nc3ccccc3)cc2)c2c(sc(C)c2C)-n2c(C)nnc21] | (1-8) |
| 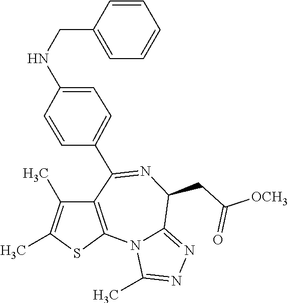 [SMILES: COC(=O)C[C@@H]1N=C(c2ccc(NCc3ccccc3)cc2)c2c(sc(C)c2C)-n2c(C)nnc21] | (1-9) |
| 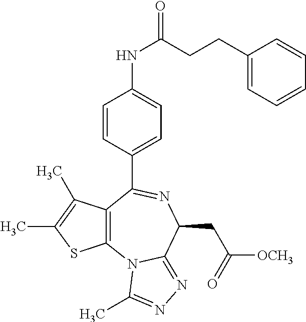 [SMILES: COC(=O)C[C@@H]1N=C(c2ccc(NC(=O)CCc3ccccc3)cc2)c2c(sc(C)c2C)-n2c(C)nnc21] | (1-10) |
| 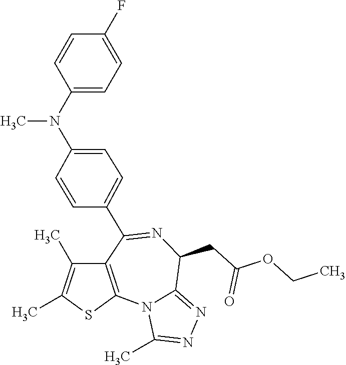 [SMILES: CCOC(=O)C[C@@H]1N=C(c2ccc(N(C)c3ccc(F)cc3)cc2)c2c(sc(C)c2C)-n2c(C)nnc21] | (1-11) |
| 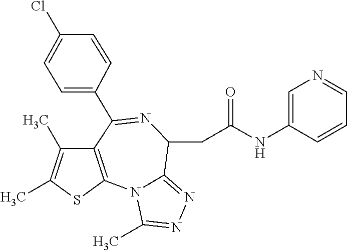 [SMILES: Cc1sc2c(c1C)C(c1ccc(Cl)cc1)=NC(CC(=O)Nc1cccnc1)c1nnc(C)n1-2] | (1-12) |
| 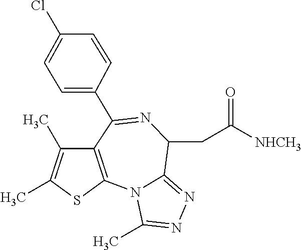 [SMILES: CNC(=O)CC1N=C(c2ccc(Cl)cc2)c2c(sc(C)c2C)-n2c(C)nnc21] | (1-13) |
| 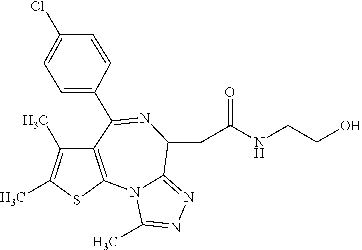 [SMILES: Cc1sc2c(c1C)C(c1ccc(Cl)cc1)=NC(CC(=O)NCCO)c1nnc(C)n1-2] | (1-14) |
| 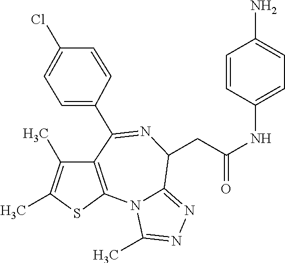 [SMILES: Cc1sc2c(c1C)C(c1ccc(Cl)cc1)=NC(CC(=O)Nc1ccc(N)cc1)c1nnc(C)n1-2] | (1-15) |
| 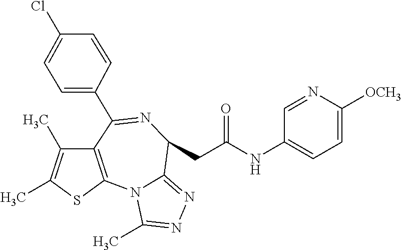 [SMILES: COc1ccc(NC(=O)C[C@@H]2N=C(c3ccc(Cl)cc3)c3c(sc(C)c3C)-n3c(C)nnc32)cn1] | (1-16) |
| 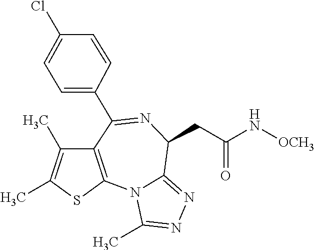 [SMILES: CONC(=O)C[C@@H]1N=C(c2ccc(Cl)cc2)c2c(sc(C)c2C)-n2c(C)nnc21] | (1-17) |
| 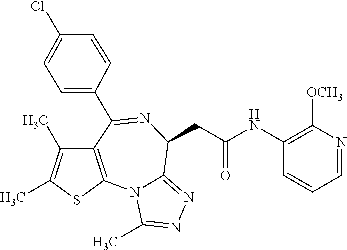 [SMILES: COc1ncccc1NC(=O)C[C@@H]1N=C(c2ccc(Cl)cc2)c2c(sc(C)c2C)-n2c(C)nnc21] | (1-18) |

In some embodiments, thienotriazolodiazepine compounds of Formula (1) include (i) (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide or a dihydrate thereof, (ii) methyl (S)-{4-(3′-cyanobiphenyl-4-yl)-2,3,9-trimethyl-6H-thieno[3,2-f][1,2,4]tri-azolo[4,3-a][1,4]diazepin-6-yl}acetate, (iii) methyl (S)-{2,3,9-trimethyl-4-(4-phenylaminophenyl)-6H-thieno[3,2-f][1,2,4]triaz-olo[4,3-a][1,4]diazepin-6-yl}acetate; and (iv) methyl (S)-{2,3,9-trimethyl-4-[4-(3-phenylpropionylamino)phenyl]-6H-thieno[3,2-f-][1,2,4]triazolo[4,3-a][1,4]diazepin-6-yl}acetate.

In some embodiments, thienotriazolodiazepine compounds of Formula (1) include (S)-2-[4-(4-chlorophenyl)-2,3,9-trimethyl-6H-thieno[3,2-f][1,2,-4]triazolo[4,3-a][1,4]diazepin-6-yl]-N-(4-hydroxyphenyl)acetamide dihydrate.

In some embodiments, thienotriazolodiazepine compounds of Formula (1) include (S)-2-[4-(4-chlorophenyl)-2,3,9-trimethyl-6H-thieno[3,2-f][1,2,-4]triazolo[4,3-a][1,4]diazepin-6-yl]-N-(4-hydroxyphenyl)acetamide.

### IV. FORMULATIONS

The compound of Formula (1) presents highly specific difficulties in relation to administration generally and the preparation of galenic compositions in particular, including the particular problems of drug bioavailability and variability in inter- and intra-patient dose response, necessitating development of a non-conventional dosage form with respect to the practically water-insoluble properties of the compound.

Previously, it had been found that the compound of Formula (1) could be formulated as a solid dispersion with the carrier ethyl acrylate-methyl methacrylate-trimethylammonioethyl methacrylate chloride copolymer (Eudragit RS, manufactured by Rohm) to provide an oral formulation that preferentially released the pharmaceutical ingredient in the lower intestine for treatment of inflammatory bowel diseases such as ulcerative colitis and Crohn's disease (US Patent Application 20090012064 A1, published Jan. 8, 2009). It was found, through various experiments, including animal tests, that in inflammatory bowel diseases drug release in a lesion and a direct action thereof on the inflammatory lesion were more important than the absorption of the drug into circulation from the gastrointestinal tract.

It has now been unexpectedly found that thienotriazolodiazepine compounds, according to Formula (1), pharmaceutically acceptable salts, solvates, including hydrates, racemates, enantiomers isomers, and isotopically-labeled forms thereof, can be formulated as a solid dispersion with pharmaceutically acceptable polymers to provide an oral formulation that provides high absorption of the pharmaceutical ingredient into the circulation from the gastrointestinal tract for treatment of diseases other than inflammatory bowel diseases. Studies in both dogs and humans have confirmed high oral bioavailability of these solid dispersions compared with the Eudragit solid dispersion formulation previously developed for the treatment of inflammatory bowel disease.

Solid dispersions are a strategy to improve the oral bioavailability of poorly water soluble drugs.

The term “solid dispersion” as used herein refers to a group of solid products including at least two different components, generally a hydrophilic carrier and a hydrophobic drug, the thienotriazolodiazepine compounds, according to Formula (1). Based on the drug's molecular arrangement within the dispersion, six different types of solid dispersions can be distinguished. Commonly, solid dispersions are classified as simple eutectic mixtures, solid solutions, glass solution and suspension, and amorphous precipitations in a crystalline carrier. Moreover, certain combinations can be encountered, for example, in the same sample some molecules may be present in clusters while some are molecularly dispersed.

In one embodiment, the thienotriazolodiazepine compounds, according to Formula (1) can be dispersed molecularly, in amorphous particles (clusters). In another embodiment, the thienotriazolodiazepine compounds, according to Formula (1) can be dispersed as crystalline particles. In one embodiment, the carrier can be crystalline. In another embodiment, the carrier can be amorphous.

In one embodiment, the present invention provides a pharmaceutical composition comprising a solid dispersion of a thienotriazolodiazepine compound, in accordance with Formula (1), or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is hypromellose acetate succinate (also called hydroxypropylmethylcellulose acetate succinate or HPMCAS). In one embodiment, the dispersion has a thienotriazolodiazepine compound to hydroxypropylmethylcellulose acetate succinate (HPMCAS) weight ratio of 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1). In some embodiments, the hydroxypropylmethyl cellulose acetate succinates (HPMCAS), may include M grade having 9% acetyl/11% succinoyl (e.g., HPMCAS having a mean particle size of 5 (i.e., HPMCAS-MF, fine powder grade) or having a mean particle size of 1 mm (i.e., HPMCAS-MG, granular grade)), H grade having 12% acetyl/6% succinoyl (e.g., HPMCAS having a mean particle size of 5 μm (i.e., HPMCAS-HF, fine powder grade) or having a mean particle size of 1 mm (i.e., HPMCAS-HG, granular grade)), and L grade having 8% acetyl/15% succinoyl (e.g., HPMCAS having a mean particle size of 5 μm (i.e., HPMCAS-LF, fine powder grade) or having a mean particle size of 1 mm (i.e., HPMCAS-LG, granular grade).

In one embodiment, the present invention provides a pharmaceutical composition comprising a solid dispersion of a thienotriazolodiazepine compound of Formula (1) or a pharmaceutically acceptable salt, a solvate, including a hydrate, a racemate, an enantiomer, an isomer, or an isotopically-labeled form thereof in a pharmaceutically acceptable polymer. In one embodiment, the pharmaceutically acceptable polymer is polyvinylpyrrolidone (also called povidone or PVP). In one embodiment, the dispersion has a thienotriazolodiazepine compound to PVP weight ratio of 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 175° C. to about 185° C. In other such embodiments, the single Tg occurs at about 179° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1). In some embodiments, the polyvinyl pyrrolidones may have molecular weights of about 2,500 (Kollidon®12 PF, weight-average molecular weight between 2,000 to 3,000), about 9,000 (Kollidon® 17 PF, weight-average molecular weight between 7,000 to 11,000), about 25,000 (Kollidon® 25, weight-average molecular weight between 28,000 to 34,000), about 50,000 (Kollidon® 30, weight-average molecular weight between 44,000 to 54,000), and about 1,250,000 (Kollidon® 90 or Kollidon® 90F, weight-average molecular weight between 1,000,000 to 1,500,000).

In one embodiment, a pharmaceutical composition of the present invention comprises a solid dispersion of an amorphous form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of thienotriazolodiazepine compound of Formula (1) to hypromellose acetate succinate ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a solid dispersion of an amorphous form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of thienotriazolodiazepine compound of Formula (1) to polyvinylpyrrolidone ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 175° C. to about 185° C. In other such embodiments, the single Tg occurs at about 179° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a solid dispersion of a crystalline form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of thienotriazolodiazepine compound of Formula (1) to hypromellose acetate succinate ranges from 1:3 to 1:1.

In one embodiment, a pharmaceutical composition of the present invention comprises a solid dispersion of a crystalline form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of thienotriazolodiazepine compound of Formula (1) to polyvinylpyrrolidone ranges from 1:3 to 1:1.

In some embodiments, a pharmaceutical composition comprising a solid dispersion is prepared by spray drying.

In one embodiment, a pharmaceutical composition of the present invention comprises a spray dried solid dispersion of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of compound (1) to hypromellose acetate succinate ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a spray dried solid dispersion of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of compound (1) to polyvinylpyrrolidone ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 175° C. to 185° C. In other such embodiments, the single Tg occurs at about 179° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a spray dried solid dispersion of an amorphous form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of thienotriazolodiazepine compound of Formula (1) to hypromellose acetate succinate ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 130° C. to 140° C. In some such embodiments, the solid dispersion was exposed to a relative humidity of 75% at 40° C. for at least one month. In other such embodiments, the single Tg occurs at about 135° C. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a spray dried solid dispersion of an amorphous form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of thienotriazolodiazepine compound of Formula (1) to polyvinylpyrrolidone ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 175° C. to 185° C. In some such embodiments, the solid dispersion was exposed to a relative humidity of 75% at 40° C. for at least one month. In other such embodiments, the single Tg occurs at about 179° C.

In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a spray dried solid dispersion of a crystalline form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of thienotriazolodiazepine compound of Formula (1) to hypromellose acetate succinate ranges from 1:3 to 1:1.

In one embodiment, a pharmaceutical composition of the present invention comprises a spray dried solid dispersion of a crystalline form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of thienotriazolodiazepine compound of Formula (1) to polyvinylpyrrolidone ranges from 1:3 to 1:1.

In one preferred embodiment, the present invention provides a pharmaceutical composition comprising a solid dispersion of 2-[(6S)-4-(4-chlorophenyl)-2,3,9-trimethyl-6H-thienol[3,2-f]-[1,2,4]triazolo[4,3-a][1,4]diazepin-6-yl]-N-(4-hydroxyphenyl)acetamide dihydrate, compound (1-1):

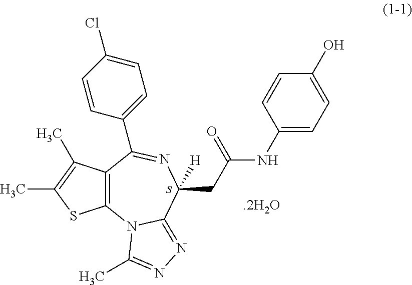

or a pharmaceutically acceptable salt, a solvate, including a hydrate, a racemate, an enantiomer, an isomer, or an isotopically-labeled form and a pharmaceutically acceptable polymer. In one embodiment, the pharmaceutically acceptable polymer is HPMCAS. In one embodiment, the dispersion has compound (1-1) and HPMCAS in a weight ratio of 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In one embodiment, the solid dispersion is spray dried. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound (1-1). For the purpose of this application “substantially free” shall mean the absence of a diffraction line, above the amorphous halo, at about 21° 2-theta associated with crystalline thienotriazolodiazepine compound (1-1).

In another embodiment, the pharmaceutical composition comprises a solid dispersion compound (1-1) or a pharmaceutically acceptable salt, a solvate, including a hydrate, a racemate, an enantiomer, an isomer, or an isotopically-labeled form; and a pharmaceutically acceptable polymer. In one embodiment, the pharmaceutically acceptable polymer is PVP. In one embodiment, the dispersion has compound (1-1) and PVP in a weight ratio 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In one embodiment, the solid dispersion is spray dried. In some embodiments, the solid dispersion exhibits a single inflection for the glass transition temperature (Tg). In some embodiments, the single Tg occurs between 175° C. to 185° C. In other such embodiments, the single Tg occurs at about 179° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound (1-1). For the purpose of this application “substantially free” shall mean the absence of a diffraction line, above the amorphous halo, at about 21° 2-theta associated with crystalline thienotriazolodiazepine compound (1-1).

In one embodiment, a pharmaceutical composition of the present invention comprises a solid dispersion of an amorphous form of a thienotriazolodiazepine compound (1-1) or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is HPMCAS. In one embodiment, the dispersion has compound (1-1) and HPMCAS in a weight ratio of 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In one embodiment, the solid dispersion is spray dried. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound (1-1). For the purpose of this application “substantially free” shall mean the absence of a diffraction line, above the amorphous halo, at about 21° 2-theta associated with crystalline thienotriazolodiazepine compound (1-1).

In one embodiment, a pharmaceutical composition of the present invention comprises a solid dispersion of an amorphous form of a thienotriazolodiazepine compound (1-1) or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is PVP. In one embodiment, the dispersion has compound (1-1) and PVP in a weight ratio 11 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In one embodiment, the solid dispersion is spray dried. In some embodiments, the solid dispersion exhibits a single inflection for the glass transition temperature (Tg). In some embodiments, the single Tg occurs between 175° C. to 185° C. In other such embodiments, the single Tg occurs at about 189° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound (1-1). For the purpose of this application “substantially free” shall mean the absence of a diffraction line, above the amorphous halo, at about 21° 2-theta associated with crystalline thienotriazolodiazepine compound (1-1).

In one embodiment, a pharmaceutical composition of the present invention comprises a solid dispersion of a crystalline form of a thienotriazolodiazepine compound (1-1) or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is HPMCAS. In one embodiment, the dispersion has compound (1-1) and HPMCAS in a weight ratio of 1:3 to 1:1. In one embodiment, the solid dispersion is spray dried.

In one embodiment, a pharmaceutical composition of the present invention comprises a solid dispersion of a crystalline form of a thienotriazolodiazepine compound (1-1) or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is PVP. In one embodiment, the dispersion has compound (1-1) and PVP in a weight ratio 1:3 to 1:1. In one embodiment, the solid dispersion is spray dried.

The solid dispersions of the invention, described herein, exhibit especially advantageous properties when administered orally. Examples of advantageous properties of the solid dispersions include, but are not limited to, consistent and high level of bioavailability when administered in standard bioavailability trials in animals or humans. The solid dispersions of the invention can include a solid dispersion comprising thienotriazolodiazepine compound of Formula (1) and a polymer and additives. In some embodiments, the solid dispersions can achieve absorption of the thienotriazolodiazepine compound of Formula (1) into the bloodstream that cannot be obtained by merely admixing the thienotriazolodiazepine compound of Formula (1) with additives since the thienotriazolodiazepine compound of Formula (1) drug has negligible solubility in water and most aqueous media. The bioavailability, of thienotriazolodiazepine compound of Formula (1) or of thienotriazolodiazepine compound (1-1) may be measured using a variety of in vitro and/or in vivo studies. The in vivo studies may be performed, for example, using rats, dogs or humans.

The bioavailability may be measured by the area under the curve (AUC) value obtained by plotting a serum or plasma concentration, of the thienotriazolodiazepine compound of Formula (1) or thienotriazolodiazepine compound (1-1), along the ordinate (Y-axis) against time along the abscissa (X-axis). The AUC value of the thienotriazolodiazepine compound of Formula (1) or thienotriazolodiazepine compound (1-1) from the solid dispersion, is then compared to the AUC value of an equivalent concentration of crystalline thienotriazolodiazepine compound of Formula (1) or crystalline thienotriazolodiazepine compound (1-1) without polymer. In some embodiments, the solid dispersion provides an area under the curve (AUC) value, when administered orally to a dog, that is selected from: at least 0.4 times, 0.5 times, 0.6 time, 0.8 time, 1.0 times, a corresponding AUC value provided by a control composition administered intravenously to a dog, wherein the control composition comprises an equivalent quantity of a crystalline thienotriazolodiazepine compound of Formula I.

The bioavailability may be measured by in vitro tests simulating the pH values of a gastric environment and an intestine environment. The measurements may be made by suspending a solid dispersion of the thienotriazolodiazepine compound of Formula (1) or thienotriazolodiazepine compound (1-1), in an aqueous in vitro test medium having a pH between 1.0 to 2.0, and the pH is then adjusted to a pH between 5.0 and 7.0, in a control in vitro test medium. The concentration of the amorphous thienotriazolodiazepine compound of Formula (1) or amorphous thienotriazolodiazepine compound (1-1) may be measured at any time during the first two hours following the pH adjustment. In some embodiments, the solid dispersion provides a concentration, of the amorphous thienotriazolodiazepine compound of Formula (1) or amorphous thienotriazolodiazepine compound (1-1), in an aqueous in vitro test medium at pH between 5.0 to 7.0 that is selected from: at least 5-fold greater, at least 6 fold greater, at least 7 fold greater, at least 8 fold greater, at least 9 fold greater or at least 10 fold greater, compared to a concentration of a crystalline thienotriazolodiazepine compound of Formula (1) or crystalline thienotriazolodiazepine compound (1-1), without polymer.

In other embodiments, the concentration of the amorphous thienotriazolodiazepine compound of Formula (1) or amorphous thienotriazolodiazepine compound (1-1), from the solid dispersion placed in an aqueous in vitro test medium having a pH of 1.0 to 2.0, is: at least 40%, at least 50% higher, at least 60%, at least 70%; at least 80%, than a concentration of a crystalline thienotriazolodiazepine compound of Formula (1) without polymer. In some such embodiments, the polymer of the solid dispersion is HPMCAS. In some such embodiments, the polymer of the solid dispersion is PVP.

In other embodiments, a concentration of the amorphous thienotriazolodiazepine compound of Formula (1) or amorphous thienotriazolodiazepine compound (1-1), from the solid dispersion, is: at least 40%, at least 50% higher, at least 60%, at least 70%; at least 80%, compared to a concentration of thienotriazolodiazepine compound of Formula (1), from a solid dispersion of thienotriazolodiazepine compound of the Formula (1) and a pharmaceutically acceptable polymer selected from the group consisting of: hypromellose phthalate and ethyl acrylate-methyl methacrylate-trimethylammonioethyl methacrylate chloride copolymer, wherein each solid dispersion was placed in an aqueous in vitro test medium having a pH of 1.0 to 2.0. In some such embodiments, the polymer of the solid dispersion is HPMCAS. In some such embodiments, the polymer of the solid dispersion is PVP.

In some embodiments, the solid dispersions, described herein, exhibit stability against recrystallization of the thienotriazolodiazepine compound of the Formula (1) or the thienotriazolodiazepine compound (1-1) when exposed to humidity and temperature over time. In one embodiment, the concentration of the amorphous thienotriazolodiazepine compound of the Formula (1) or the thienotriazolodiazepine compound (1-1) which remains amorphous is selected from: at least 90%, at least 91%, at least 92%, at least 93%, at least 94%, at least 95%, at least 96%, at least 97%, at least 98% and at least 99%.

### V. DOSAGE FORMS

Suitable dosage forms that can be used with the solid dispersions of the present invention include, but are not limited to, capsules, tablets, mini-tablets, beads, beadlets, pellets, granules, granulates, and powder. Suitable dosage forms may be coated, for example using an enteric coating. Suitable coatings may comprise but are not limited to cellulose acetate phthalate, hydroxypropylmethylcellulose (HPMC), hydroxypropylmethylcellulose phthalate, a polymethylacrylic acid copolymer, or hydroxylpropylmethylcellulose acetate succinate (HPMCAS). In some embodiments, certain combinations can be encountered, for example, in the same sample some molecules of the thienotriazolodiazepine of the present invention may be present in clusters while some are molecularly dispersed with a carrier.

In some embodiments, the solid dispersions of the invention may be formulated as tablets, caplets, or capsules. In one some embodiments, the solid dispersions of the invention may be formulated as mini-tablets or pour-into-mouth granules, or oral powders for constitution. In some embodiments, the solid dispersions of the invention are dispersed in a suitable diluent in combination with other excipients (e.g., re-crystallization/precipitation inhibiting polymers, taste-masking components, etc) to give a ready-to-use suspension formulation. In some embodiments, the solid dispersions of the invention may be formulated for pediatric treatment.

In one embodiment, the pharmaceutical composition of the present invention is formulated for oral administration. In one embodiment, the pharmaceutical composition comprises a solid dispersion, according to the various embodiments described herein, comprising a thienotriazolodiazepine compound of Formula (1) or a pharmaceutically acceptable salt, a solvate, including a hydrate, a racemate, an enantiomer, an isomer, or an isotopically-labeled form thereof; and a polymer carrier. In one embodiment, the pharmaceutical composition further includes one or more additives such as disintegrants, lubricants, glidants, binders, and fillers.

Examples of suitable pharmaceutically acceptable lubricants and pharmaceutically acceptable glidants for use with the pharmaceutical composition include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose, glyceryl behenate, stearic acid, hydrogenated castor oil, glyceryl monostearate, and sodium stearyl fumarate.

Examples of suitable pharmaceutically acceptable binders for use with the pharmaceutical composition include, but are not limited to starches; celluloses and derivatives thereof, e.g., microcrystalline cellulose (e.g., AVICEL PH from FMC), hydroxypropyl cellulose, hydroxyethyl cellulose, and hydroxylpropylmethylcellulose (HPMC, e.g., METHOCEL from Dow Chemical); sucrose, dextrose, corn syrup; polysaccharides; and gelatin.

Examples of suitable pharmaceutically acceptable fillers and pharmaceutically acceptable diluents for use with the pharmaceutical composition include, but are not limited to, confectioner's sugar, compressible sugar, dextrates, dextrin, dextrose, lactose, mannitol, microcrystalline cellulose (MCC), powdered cellulose, sorbitol, sucrose, and talc.

In some embodiments, excipients may serve more than one function in the pharmaceutical composition. For example, fillers or binders may also be disintegrants, glidants, anti-adherents, lubricants, sweeteners and the like.

In some embodiments, the pharmaceutical compositions of the present invention may further include additives or ingredients, such as antioxidants (e.g., ascorbyl palmitate, butylated hydroxylanisole (BHA), butylated hydroxytoluene (BHT), α-tocopherols, propyl gallate, and fumaric acid), antimicrobial agents, enzyme inhibitors, stabilizers (e.g., malonic acid), and/or preserving agents.

Generally, the pharmaceutical compositions of the present invention may be formulated into any suitable solid dosage form. In some embodiments, the solid dispersions of the invention are compounded in unit dosage form, e.g., as a capsule, or tablet, or a multi-particulate system such as granules or granulates or a powder, for administration.

In one embodiment, a pharmaceutical compositions includes a solid dispersion of a thienotriazolodiazepine compound of Formula (1), according to the various embodiments of solid dispersions described herein, and hydroxypropylmethylcellulose acetate succinate (HPMCAS), wherein the thienotriazolodiazepine compound is amorphous in the solid dispersion and has a thienotriazolodiazepine compound to hydroxypropylmethylcellulose acetate succinate (HPMCAS), weight ratio of 1:3 to 1:1; 45-50 wt. % of lactose monohydrate; 35-40 wt. % of microcrystalline cellulose; 4-6 wt. % of croscarmellose sodium; 0.8-1.5 wt. % of colloidal silicon dioxide; and 0.8-1.5 wt. % of magnesium stearate.

### VI. DOSAGE

In one embodiment, the present invention provides a pharmaceutical composition that maybe formulated into any suitable solid dosage form. In one embodiment, a pharmaceutical composition in accordance with the present invention comprises one or more of the various embodiments of the thienotriazolodiazepine of Formula (1) as described herein in a dosage amount ranging from about 10 mg to about 100 mg. In one embodiment, the pharmaceutical composition of the present invention includes one or more of the various embodiments of the thienotriazolodiazepine of Formula (1) as described herein in a dosage amount selected from the group consisting of from about 10 mg to about 100 mg, about 10 mg to about 95 mg, about 10 mg to about 90 mg, about 10 mg to about 85 mg, about 10 mg to about 80 mg, about 10 mg to about 75 mg, about 10 mg to about 70 mg, about 10 mg to about 65 mg, about 10 mg to about 60 mg, about 10 mg to about 55 mg, about 10 mg to about 50 mg, about 10 mg to about 45 mg, about 10 mg to about 40 mg, about 10 mg to about 35 mg, about 10 mg to about 30 mg, about 10 mg to about 25 mg, about 10 mg to about 20 mg, and about 10 mg to about 15 mg. In one embodiment, the pharmaceutical composition of the present invention includes one or more of the various embodiments of the thienotriazolodiazepine of Formula (1) as described herein in a dosage amount selected from the group consisting of about 10 mg, about 50 mg, about 75 mg, about 100 mg.

In some embodiments, the methods of the present invention includes administering to a subject in need thereof one or more of the various embodiments of the thienotriazolodiazepine of Formula (I) as described herein in a dosage amount selected from the group consisting of about 1 mg, about 2 mg, about 2.5 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, and about 150 mg, and in a dosage form selected from the group consisting of once weekly, once daily every sixth day, once daily every fifth day, once daily every fourth day, once daily every third day, once daily every other day, once daily, twice daily, three times daily, four times daily, and five times daily. In another embodiment, any of the foregoing dosage amounts or dosage forms is decreased periodically or increased periodically.

In some embodiments, the methods of the present invention includes administering to a subject in need thereof a thienotriazolodiazepine selected from the group consisting of compounds (1-1), (1-2), (1-3), (1-4), (1-5), (1-6), (1-7), (1-8), (1-9), (1-10), (1-11), (1- 12), (1-13), (1-14), (1-15), (1-16), (1-17), and (1-18), in a dosage amount selected from the group consisting of about 1 mg, about 2 mg, about 2.5 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, and about 150 mg, and in a dosage form selected from the group consisting of once weekly, once daily every sixth day, once daily every fifth day, once daily every fourth day, once daily every third day, once daily every other day, once daily, twice daily, three times daily, four times daily, and five times daily. In another embodiment, any of the foregoing dosage amounts or dosage forms is decreased periodically or increased periodically.

Such unit dosage forms are suitable for administration 1 to 5 times daily depending on the particular purpose of therapy, the phase of therapy, and the like. In one embodiment, the dosage form may be administered to a subject in need thereof at least once daily for at least two successive days. In one embodiment, the dosage form may be administered to a subject in need thereof at least once daily on alternative days. In one embodiment, the dosage form may be administered to a subject in need thereof at least weekly and divided into equal and/or unequal doses. In one embodiment, the dosage form may be administered to a subject in need thereof weekly, given either on three alternate days and/or 6 times per week. In one embodiment, the dosage form may be administered to a subject in need thereof in divided doses on alternate days, every third day, every fourth day, every fifth day, every sixth day and/or weekly. In one embodiment, the dosage form may be administered to a subject in need thereof two or more equally or unequally divided doses per month.

The dosage form used, e.g., in a capsule, tablet, mini-tablet, beads, beadlets, pellets, granules, granulates, or powder may be coated, for example using an enteric coating. Suitable coatings may comprise but are not limited to cellulose acetate phthalate, hydroxypropylmethylcellulose (HPMC), hydroxypropylmethylcellulose phthalate, a polymethylacrylic acid copolymer, or hydroxylpropylmethylcellulose acetate succinate (HPMCAS).

### VII. PROCESS

The thienotriazolodiazepine compounds disclosed herein can exist as free base or as acid addition salt can be obtained according to the procedures described in US Patent Application Publication No. 2010/0286127, incorporated by reference in its entirety herein, or in the present application. Individual enantiomers and diastereomers of the thienotriazolodiazepine compounds of the present invention can be prepared synthetically from commercially available starting materials that contain asymmetric or stereogenic centers, or by preparation of racemic mixtures followed by resolution methods well known to those of ordinary skill in the art.

In some embodiments, a one or more of the various embodiments for the formulation of the thienotriazolodiazepine, according to Formula (1), is prepared by a solvent evaporation method. In one embodiment, the solvent evaporation method comprises solubilization of a thienotriazolodiazepine compound, according to Formula (1), carrier in a volatile solvent that is subsequently evaporated. In one embodiment, the volatile solvent may one or more excipients. In one embodiment, the one or more excipients include, but are not limited to anti-sticking agents, inert fillers, surfactants wetting agents, pH modifiers and additives. In one embodiment, the excipients may dissolved or in suspended or swollen state in the volatile solvent.

In one embodiment, preparation of solid dispersions in accordance with the present invention includes drying one or more excipients suspended in a volatile solvent. In one embodiment, the drying includes vacuum drying, slow evaporation of the volatile solvent at low temperature, use of a rotary evaporator, spray-drying, spray granulation, freeze-drying, or use of supercritical fluids.

In one embodiment, spray drying preparation of a formulation for the thienotriazolodiazepine composition, according to Formula (1), is used which involves atomization of a suspension or a solution of the composition into small droplets, followed by rapid removal solvent from the formulation. In one embodiment, preparation of a formulation in accordance with the present invention involves spray granulation in which a solution or a suspension of the composition in a solvent is sprayed onto a suitable chemically and/or physically inert filler, such as lactose or mannitol. In one embodiment, spray granulation of the solution or the suspension of the composition is achieved via two-way or three-way nozzles.

The invention is illustrated in the following non-limiting examples.

### VIII. EXAMPLES

### Example 1

### In Vitro Screening of Solid Dispersions of Compound (1-1)

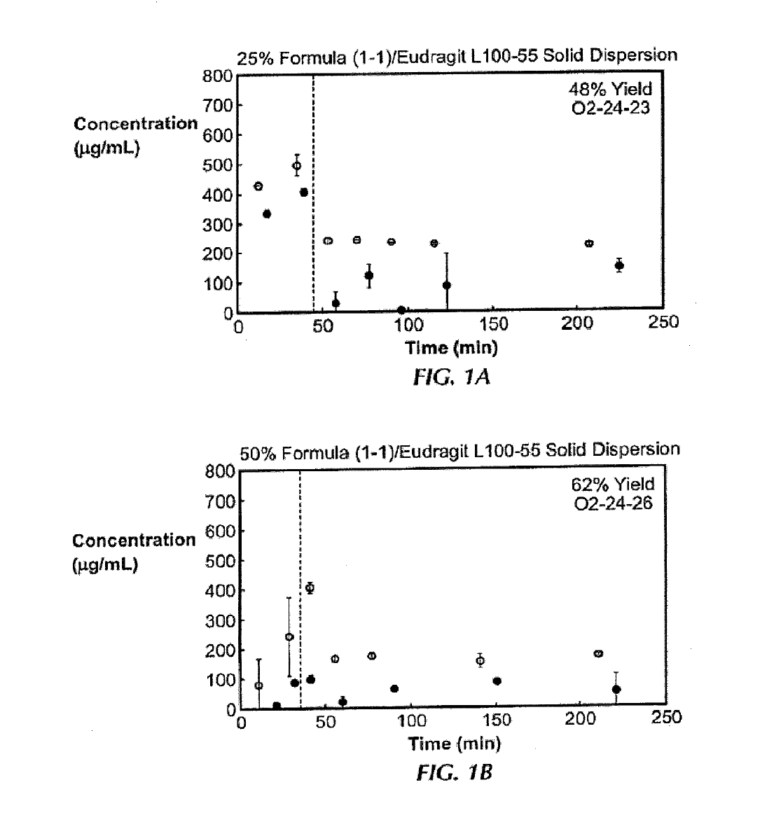

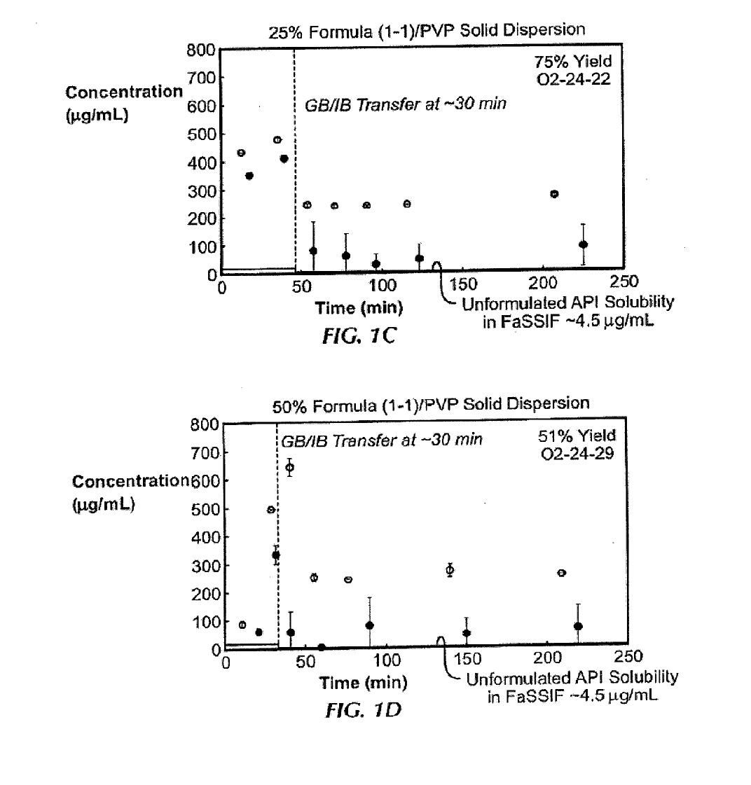

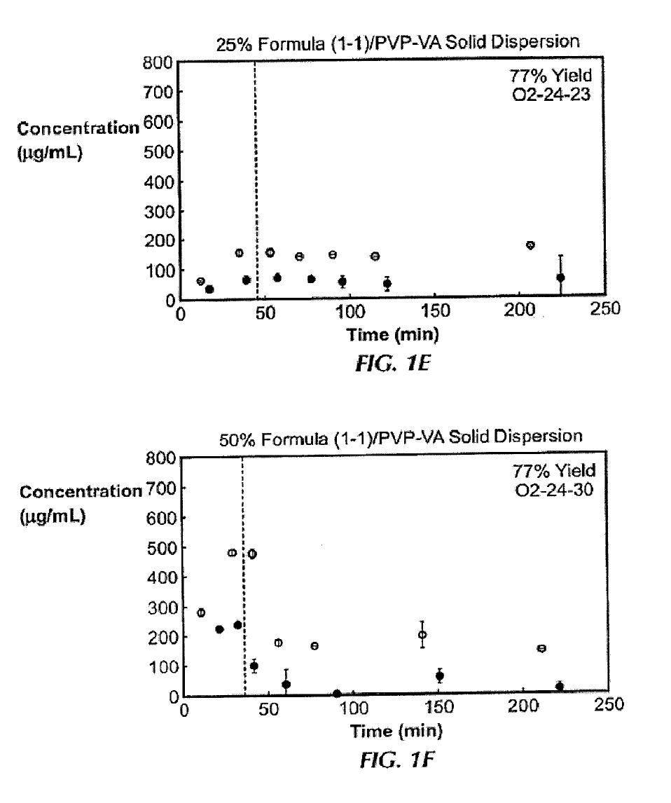

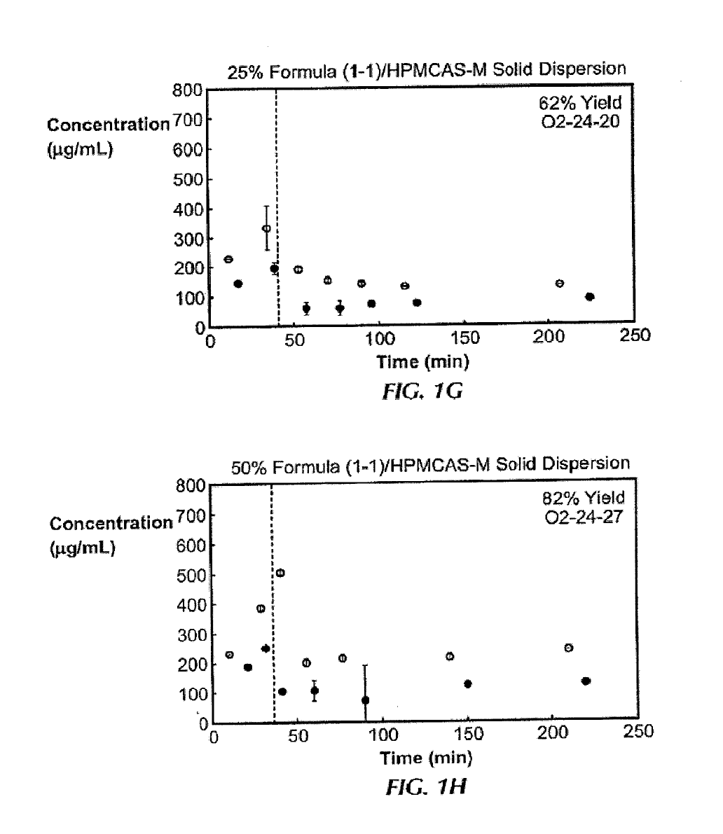

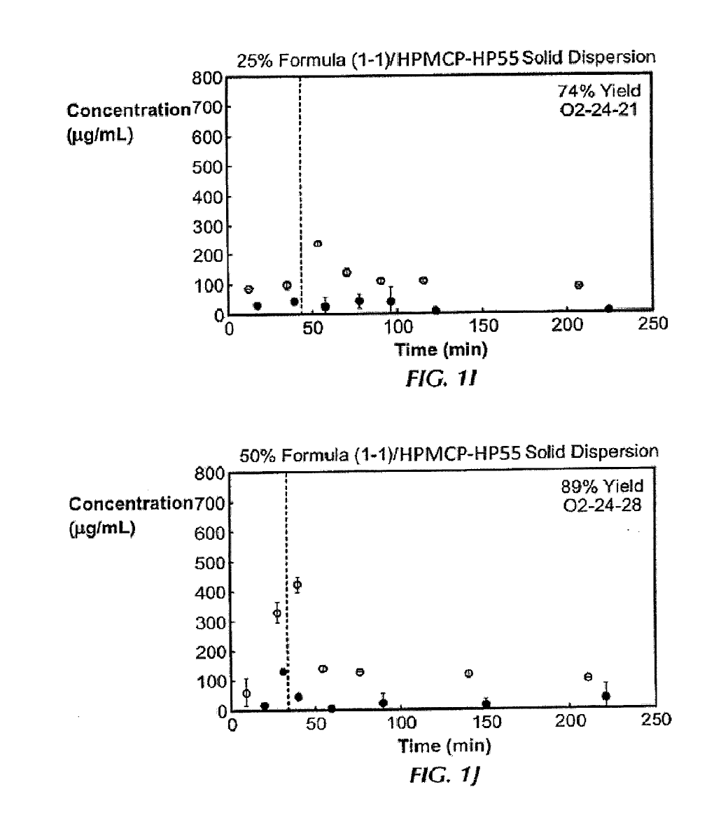

Ten solid dispersions were prepared using compound (1-1) and one of five polymers, including hypromellose acetate succinate (HPMCAS-M), hypromellose phthalate (HPMCP-HP55), polyvinylpyrrolidone (PVP), PVP-vinyl acetate (PVP-VA), and Eudragit L100-55, at both 25% and 50% of compound (1-1) loading, for each polymer. Solid dispersions were prepared by a solvent evaporation method, using spray-drying followed by secondary drying in a low-temperature convection oven. The performance of each solid dispersion was assessed via a non-sink dissolution performance test which measured both the total amount of drug and the amount of free drug present in solution over time. Non-sink dissolution was chosen because it best represents the in vivo situation for low soluble compounds. This test included a “gastric transfer” of dispersion from gastric pH (0.1N NaCl, pH 1.0) to intestinal pH (FaFSSIF, pH 6.5) approximately 30 to 40 minutes after the introduction of dispersion to the test medium, simulating in vivo conditions. [FaFSSIF is Fasted State Simulated Intestinal Fluid, comprised of 3 mM sodium taurocholate, 0.75 mM lechithin, 0.174 g NaOH pellets, 1.977 g NaH₂PO₄.H₂O, 3.093 g NaCl, and purified water qs 500 mL.] The amount of dissolved drug was quantified using a high-performance liquid chromatrography (HPLC) method and an Agilent 1100 series HPLC. The dissolution profiles of the formulations (FIGS. 1A-1J) showed large increases in drug solubility in all dispersion candidates relative to the unformulated compound in the same media. Of the solid dispersions, the 25% compound (1-1) in PVP, 25% compound (1-1) in HPMCAS-M, and 50% compound (1-1) in HPMCAS-M dispersions were the most promising candidates for enhanced oral absorption as compared to the unformulated compound, based on finding higher levels of free drug released at intestinal pH.

### Example 2

### In Vivo Screening of Solid Dispersions of Compound (1-1)

The three most promising solid dispersions of compound (1-1), namely the 25% compound (1-1) in PVP, 25% compound (1-1) in HPMCAS-MG, and 50% compound (1-1) in HPMCAS-M dispersions, were prepared at larger scale for in vivo studies. Each formulation was assessed in the in vitro dissolution test described in Example 1. To ensure that these dispersions were both amorphous and homogeneous, each dispersion was assessed by powder x-ray diffraction (PXRD) and modulated differential scanning calorimetry (mDSC). Additionally, to understand the effect of water on the glass transition temperature (Tg) for each dispersion, mDSC was performed on samples first equilibrated at a set relative humidity (i.e., 25%, 50%, and 75% RH) for at least 18 hours. [Water can act as a plasticizer for solid dispersions and the hygroscopicity of the system due to the active compound or polymer can affect the amount of water uptake by these systems.]

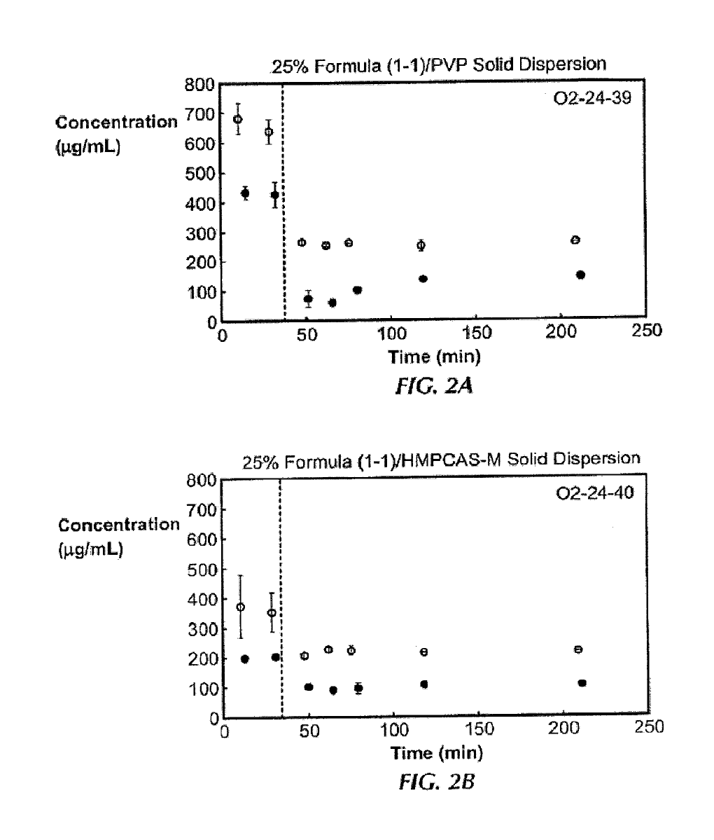

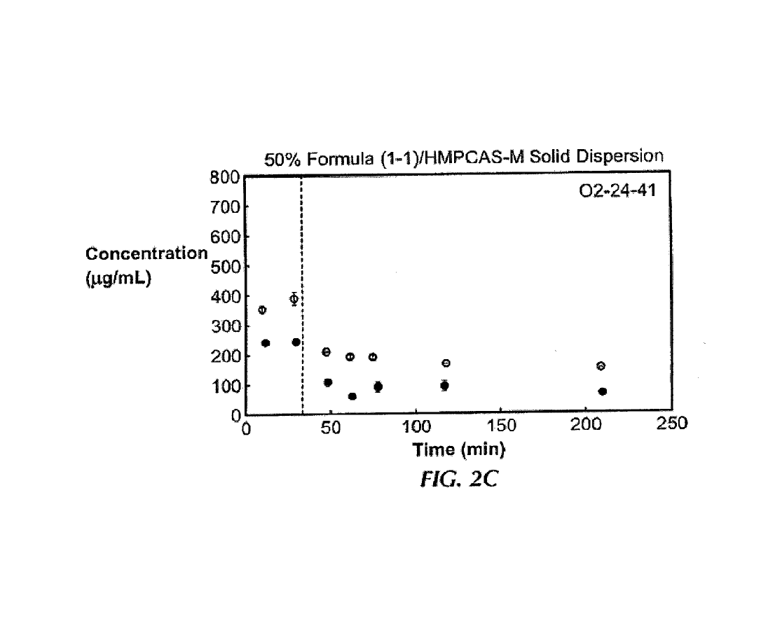

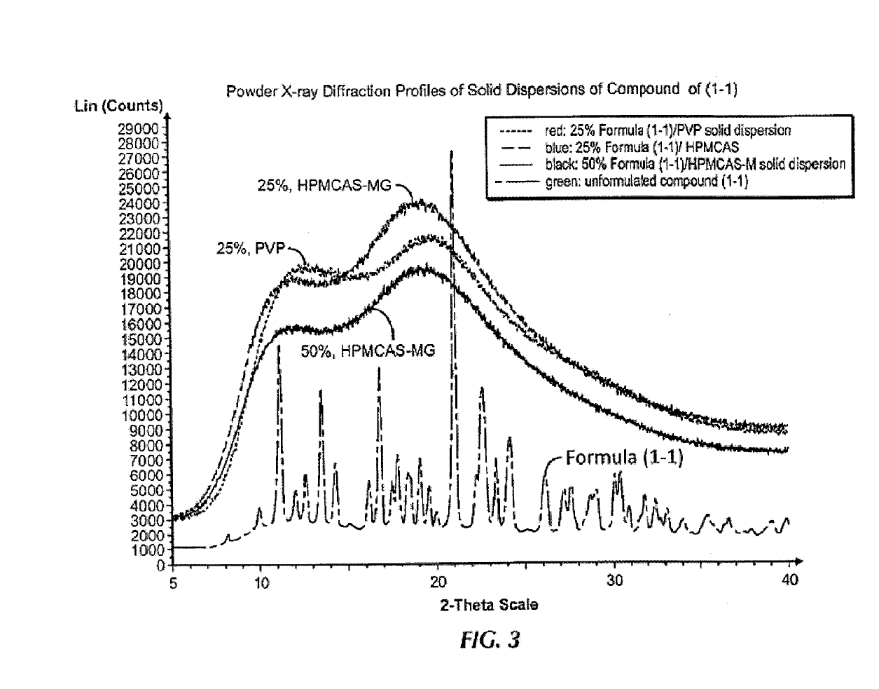

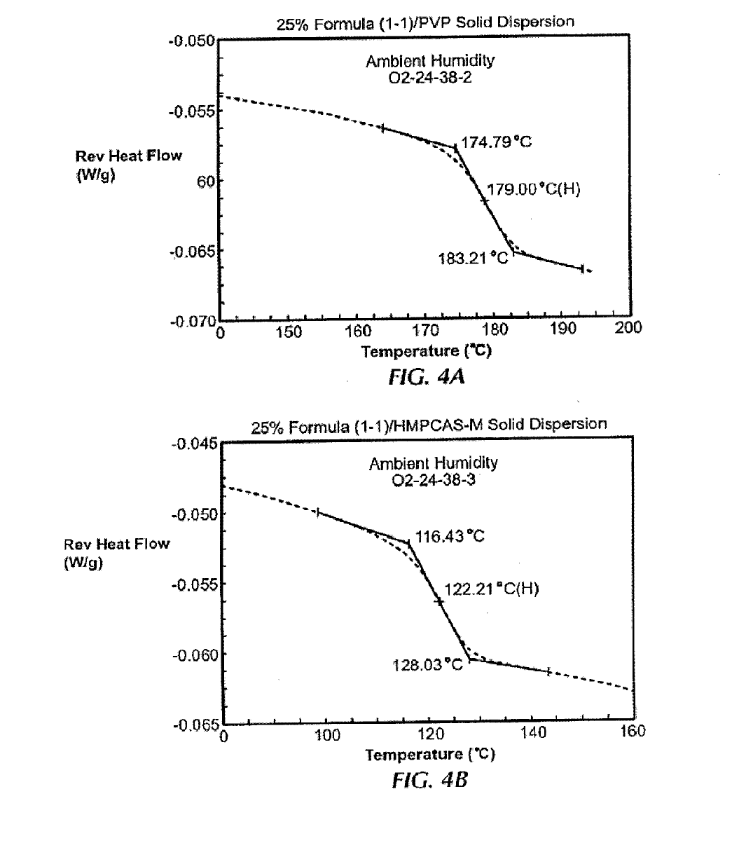

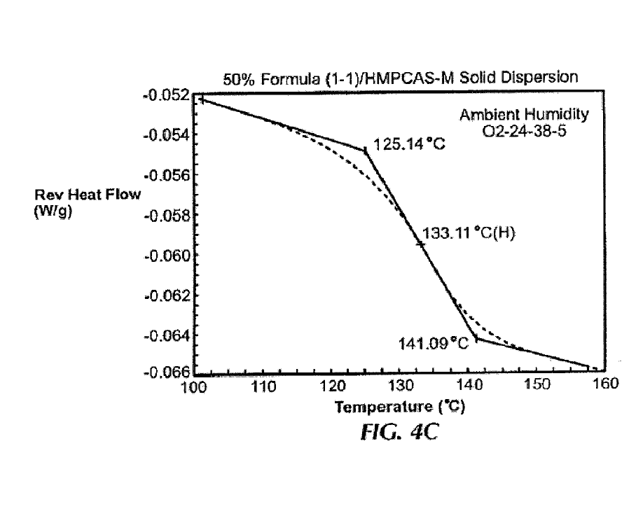

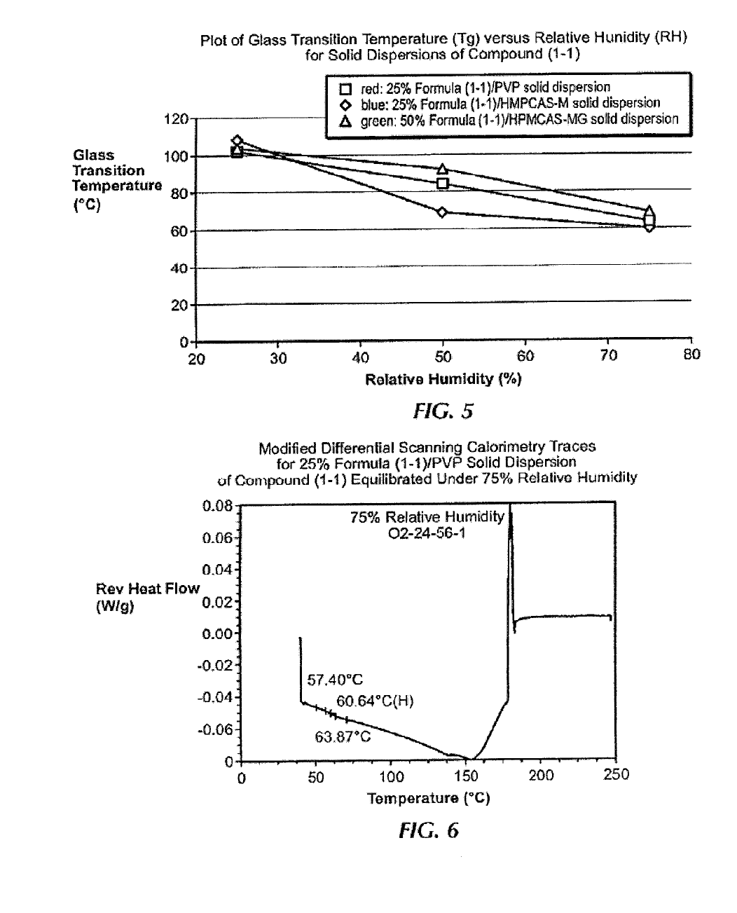

The non-sink dissolution results (FIGS. 2A-2C) were comparable to those found for the dispersions in Example 1. PXRD results (FIG. 3) showed no evidence of crystalline compound in any of the dispersions and mDSC results (FIGS. 4A-4C) showed a single glass transition temperature (Tg) for each dispersion, indicating that each dispersion was homogeneous. The x-ray diffractomer was a Bruker D-2 Phaser. An inverse relationship between Tg and relative humidity was observed for each (FIG. 5). Notably, for the 25% compound (1-1) in PVP solid dispersion equilibrated at 75% RH, there appeared to be two Tgs, indicating that phase separation was occurring, and this dispersion also showed a melt event at 75% RH, suggesting that crystallization occurred during the RH equilibration (FIG. 6). This finding suggests that the 25% compound (1-1) in PVP dispersion may be less stable than the HPMCAS-M dispersions.

To assess the bioavailability of the three dispersions, groups of male beagle dogs (three per group) were given a 3 mg/kg dose of an aqueous suspension of solid dispersion of compound (1-1) administered by oral gavage or a 1 mg/kg dose of compound (1-1) dissolved in water:ethanol:polyethylene glycol (PEG) 400 (60:20:20) and administered as an intravenous bolus into the cephalic vein. Blood samples were collected from the jugular vein of each animal at 0 (pre-dose), 5, 15, and 30 minutes and 1, 2, 4, 8, 12, and 24 hours following intravenous administration and at 0 (pre-dose), 15 and 30 minutes and 1, 2, 4, 8, 12, and 24 hours following oral gavage administration. The amount of compound (1-1) present in each sample was detected using a qualified LC-MS/MS method with a lower limit of quantification of 0.5 ng/mL. The area under the plasma concentration-time curve (AUC) was determined by use of the linear trapezoidal rule up to the last measurable concentration without extrapolation of the terminal elimination phase to infinity. The elimination half-life (t_{1/2}) was calculated by least-squares regression analysis of the terminal linear part of the log concentration-ime curve. The maximum plasma concentration (Cₘₐₓ) and the time to Cₘₐₓ (tₘₐₓ) were derived directly from the plasma concentration data. The oral bioavailability (F) was calculated by dividing the dose normalized AUC after oral administration by the dose normalized AUC after intravenous administration and reported as percentages (%). Results, summarized in Table 1 below, gave mean oral bioavailabilities of the 25% compound (1-1) in PVP, 25% compound (1-1) in HPMCAS-M, and 50% compound (1-1) in HPMCAS-M solid dispersions of 58%, 49%, and 74%, respectively.

TABLE 1
pharmacokinetic parameters of compound (1-1)
after oral (po) and intravenous (iv) administrations
to dogs (the values are averages from three dogs)

| Compound (1-1) | Dose & | Cₘₐₓ | tₘₐₓ | AUC | t_{1/2} | F |
|---|---|---|---|---|---|---|
| formulation | Route | (ng/L) | (hr) | (ng · min/mL) | (hr) | (%) |
| Solution in water: | 1 mg/kg | 769 | 0.083 | 53,312 | 1.5 | — |
| ethanol:PEG400 | IV | | | | | |
| (60:20:20) | | | | | | |
| Aqueous suspension | 3 mg/kg | 487 | 1.0 | 93,271 | 1.6 | 58 |
| of 25% | PO | | | | | |
| compound (1-1)/ | | | | | | |
| PVP solid | | | | | | |
| dispersion | | | | | | |
| Aqueous suspension | 3 mg/kg | 228 | 0.5 | 78,595 | 2.0 | 49 |
| of 25% | PO | | | | | |
| compound (1-1)/ | | | | | | |
| HPMCAS-M | | | | | | |
| solid dispersion | | | | | | |
| Aqueous suspension | 3 mg/kg | 371 | 1.0 | 118,174 | 1.5 | 74 |
| of 50% | PO | | | | | |
| compound (1-1)/ | | | | | | |
| HPMCAS-M | | | | | | |
| solid dispersion | | | | | | |

AUC: area under the plasma concentration-time curve;
Cₘₐₓ: maximum plasma concentration;
F: bioavailability;
HPMCAS: hypromellose acetate sodium;
IV: intravenous;
PEG: polyethylene glycon;
PO; per os, oral;
PVP: polyvinylpyrrolidone;
tₘₐₓ: time of Cₘₐₓ;
t_{1/2}: plasma elimination half-life

### Example 3

### Preparation and Clinical Use of Capsules Containing a Solid Dispersion of Compound (1-1)

A gelatin capsule of 10 mg strength was prepared for initial clinical studies in patients with hematologic malignancies. Based on results of in vitro and in vivo testing of solid dispersions of compound (1-1), as described in Examples 1 and 2, a 50% compound (1-1) in HPMCAS-M solid dispersion was selected for capsule development. Capsule development was initiated targeting a fill weight of 190 mg in a size 3 hard gelatin capsule, as this configuration would potentially allow increasing the capsule strength by filling a larger size capsule while maintaining the pharmaceutical composition. Based on experience, four capsule formulations were designed with different amounts of disintegrant and with and without wetting agent. Since all four formulations showed similar disintegration test and dissolution test results, the simplest formulation (without wetting agent and minimum disintegrant) was selected for manufacturing. Manufacturing process development and scale-up studies were performed to confirm the spray drying process and post-drying times for the solid dispersion; blending parameters; roller compaction and milling of the blend to achieve target bulk density of approximately 0.60 g/cc; and capsule filling conditions.

Crystalline compound (1-1) and the polymer hypromellose actate succinate (HPMCAS-M) were dissolved in acetone and spray-dried to produce solid dispersion intermediate (SDI) granules containing a 50% compound (1-1) loading. The SDI was shown by PXRD analysis to be amorphous and by mDSC analysis to be homogeneous (i.e., single Tg under ambient conditions). The 50% compound (1-1) in HPMCAS-M solid dispersion (1000 g) and excipients, including microcrystalline cellulose filler-binder (4428 g), croscarmellose sodium disintegrant (636 g), colloidal silicon dioxide dispersant/lubricant 156 g), magnesium stearate dispersant/lubricant (156 g), and lactose monohydrate filler (5364 g) were blended in stages in a V-blender. The blend was them compacted and granulated to obtain a bulk density of approximately 0.6 g/mL. The blend was dispensed into size 3 hard gelatin capsules (target fill weight: 190 mg) using an automated filling machine and finished capsules were polished using a capsule polisher machine.

Pharmacokinetic assessments were performed following oral dosing of 10 mg capsules containing the 50% compound (1-1) in HPMCAS solid dispersion and results were compared with pharmacokinetic assessments performed following oral dosing of administration of 4×10 mg capsules containing the Eudragit solid dispersion of compound (1-1) to healthy volunteers

A comparison of the two pharmaceutical compositions is provided in Tables 2A and 2B below. The Eudragit formulation previously was described in Example 5 in US Patent Application 2009/0012064 A1, published Jan. 8, 2009. That application noted that the Eudragit solid dispersion formulation was made by dissolving and/or dispersing the thienotriazolodiazepine of formula (A) and coating excipients, including ammonio methacrylate copolymer type B (Eudragit RS), methacrylic acid copolymer type C (Eudragit L100-55), talc, and magnesium aluminosilicate, in a mixture of water and ethanol. This heterogeneous mixture then was applied to microcrystalline cellulose spheres (Nonpareil 101, Freund) using a centrifugal fluidizing bed granulator to produce granules that were dispensed into size 2 hydroxypropyl methylcellulose capsules.

In both clinical studies, blood levels of compound (1-1) were determined using validated LC-MS/MS methods and pharmacokinetic analyses were performed based on plasma concentrations of compound (1-1) measured at various time points over 24 hours after capsule administration. Results, summarized in Table 3 below, showed that the HPMCAS-M solid dispersion formulation had over 3-fold higher bioavailability in humans than the Eudragit solid dispersion formulation based on AUCs (924*4/1140, adjusting for difference in doses administered). Additionally, based on the observed Tₘₐₓ, the HPMCAS formulation is more rapidly absorbed than the Eudragit formulation (Tₘₐₓ of 1 h vs 4-6 h). The marked improvement in systemic exposure with the HPMCAS-M solid dispersion formulation is unexpected.

TABLE 2A
solid dispersion capsules of compound (1-1) for clinical use
pharmaceutical composition containing 50% HPMCAS solid dispersion
of compound (1-1): 10 mg strength, size 3 hard gelatin capsule

| | | Capsule Content | |
|---|---|---|---|
| Ingredient | Function | mg | Wt % |
| Compound of formula (II) | active agent | 10.0* | 5.56 |
| Hypromellose acetate succinate | carrier for solid | 10.0 | 5.56 |
| (HPMCAS-M) | dispersion | | |
| Lactose monohydrate | filler | 85.0 | 47.22 |
| Microcrystalline cellulose | filler-binder | 70.0 | 38.89 |
| Croscarmellose sodium | disintegrant | 10.0 | 5.56 |
| Collidal silicon dioxide | dispersant/lubricant | 2.5 | 1.39 |
| Magnesium stearate | dispersant/lubricant | | |

| | | |
|---|---|---|
| Total | 190.0 | 100.0 |

TABLE 2B
pharmaceutical composition containing Eudragit L100-55 solid
dispersion of compound (1-1): 10 mg strength, size 2 hard
gelatin capsule

| | | Capsule Content | |
|---|---|---|---|
| Ingredient | Function | mg | Wt % |
| Compound (1-1) | active agent | 10.0* | 3.8 |
| Core: | | | |
| Microcrystalline cellulose spheres | vehicle | 100.0 | 38.5 |
| (Nonpareil 101, Freund, Inc) | | | |
| Compound/polymer layer: | | | |
| Ammonio methacrylate | coating agent | 10.8 | 4.2 |
| copolymer, type B (NF. PhEur) | | | |
| (Edragit RS, Evonik) | | | |
| Methacrylic acid copolymer, | coating agent | 25.2 | 9.7 |
| type C (NF)/ | | | |
| Methacrylic acid-ethyl acrylate | | | |
| copolymer (1:1) type A (PhEur) | | | |
| (Eudragit L100-55, Evonik) | | | |
| Talc | coating agent | 88.2 | 33.9 |
| Magnesium aluminometasilicate | coating agent | 20.0 | 7.7 |
| (Neuslin, Fuji Chemical) | | | |
| Triethyl citrate | plasticizer | 5.0 | 1.9 |
| Silicon dioxide | fluidizing agent | 0.8 | 0.3 |
| | | 260.0 | 100.0 |

*as anhydrate

TABLE 3
pharmacokinetic parameters following oral administration of solid
dispersions of compound (1-1) to humans

| | | Dose | | | |
|---|---|---|---|---|---|
| Compound (1-1) | # | and | Cₘₐₓ | Tₘₐₓ | AUC_{0-24 h} |
| formulation | Patients | Route | (ng/mL) | (hr) | (ng · h/mL) |
| Eudragit solid dispersion | 7 | 40 mg | 83 | 4 to 6 | 1140 |
| formulation | | PO | | | |
| 50% HMPCAS-M solid | 7 | 10 mg | 286 | 1 | 925 |
| dispersion formulation | | PO | | | |

AUC_{0-24 h}: area under the OTX015 plasma concentration vs. time curve over 24 hours
Cₘₐₓ: maximum concentration in plasma
hr: hour
HPMCAS: hypromellose acetate succinate
mL: milliliter
ng: nanogram
PO: per os, oral
Tₘₐₓ: time of Cₘₐₓ

### Example 4

### Oral Exposure in the Rat

The oral bioavailability of three formulations of solid dispersions of compound (1-1) was determined in rats. The three dispersions chosen were the 25% dispersion of compound (1-1) in PVP, the 25% dispersion of compound (1-1) in HPMCAS-MG, and the 50% dispersion of compound (1-1) in HPMCAS-MG. The animals used in the study were Specific Pathogen Free (SPF) Hsd:Sprague Dawley rats obtained from the Central Animal Laboratory at the University of Turku, Finland. The rats were originally purchased from Harlan, The Netherlands. The rats were female and were ten weeks of age, and 12 rats were used in the study. The animals were housed in polycarbonate Makrolon II cages (three animals per cage), the animal room temperature was 21+/−3° C., the animal room relative humidity was 55+/−15%, and the animal room lighting was artificial and was cycled for 12 hour light and dark periods (with the dark period between 18:00 and 06:00 hours). Aspen chips (Tapvei Oy, Estonia) were used for bedding, and bedding was changed at least once per week. Food and water was provided prior to dosing the animals but was removed during the first two hours after dosing.

The oral dosing solutions containing the 25% dispersion of compound (1-1) in PVP, the 25% dispersion of compound (1-1) in HPMCAS-MG, and the 50% dispersion of compound (1-1) in HPMCAS-MG were prepared by adding a pre-calculated amount of sterile water for injection to containers holding the dispersion using appropriate quantities to obtain a concentration of 0.75 mg/mL of compound (1-1). The oral dosing solutions were subjected to vortex mixing for 20 seconds prior to each dose. The dosing solution for intravenous administration contained 0.25 mg/mL of compound (1-1) and was prepared by dissolving 5 mg of compound (1-1) in a mixture containing 4 mL of polyethylene glycol with an average molecular weight of 400 Da (PEG400), 4 mL of ethanol (96% purity), and 12 mL of sterile water for injection. The dosing solution containing the 25% dispersion of compound (1-1) in PVP was used within 30 minutes after the addition of water. The dosing solutions containing the 25% dispersion of compound (1-1) in HPMCAS-MG and the 50% dispersion of compound (1-1) in HPMCAS-MG were used within 60 minutes of after the addition of water. A dosing volume of 4 mL/kg was used to give dose levels of compound (1-1) of 1 mg/kg for intravenous administration and 3 mg/kg for oral administration. The dosing scheme is given in Table 4.

TABLE 4
Dosing scheme for rat oral exposure study.

| | | Dose | | |
|---|---|---|---|---|
| Rat | Weight | (mL) | Test Item | Route |
| 1 | 236.5 | 0.95 | Compound (1-1) | intravenous |
| 2 | 221 | 0.88 | Compound (1-1) | intravenous |
| 3 | 237.5 | 0.95 | Compound (1-1) | intravenous |
| 4 | 255.5 | 1.02 | 25% dispersion of compound (1-1) | oral |
| | | | in PVP | |
| 5 | 224.2 | 0.90 | 25% dispersion of compound (1-1) | oral |
| | | | in PVP | |
| 6 | 219.2 | 0.88 | 25% dispersion of compound (1-1) | oral |
| | | | in PVP | |
| 7 | 251.6 | 1.01 | 25% dispersion of compound (1-1) | oral |
| | | | in HPMCAS-MG | |
| 8 | 240.4 | 0.96 | 25% dispersion of compound (1-1) | oral |
| | | | in HPMCAS-MG | |
| 9 | 238 | 0.95 | 25% dispersion of compound (1-1) | oral |
| | | | in HPMCAS-MG | |
| 10 | 226.6 | 0.91 | 50% dispersion of compound (1-1) | oral |
| | | | in HPMCAS-MG | |
| 11 | 228.4 | 0.91 | 50% dispersion of compound (1-1) | oral |
| | | | in HPMCAS-MG | |
| 12 | 228.5 | 0.91 | 50% dispersion of compound (1-1) | oral |
| | | | in HPMCAS-MG | |

Blood samples of approximately 50 μL were collected into Eppendorf tubes containing 5 μL of ethylenediaminetetraacetic acid (EDTA) solution at time points of 0.25, 0.5, 1, 2, 4, 8, 12, and 24 hours after dosing, with each sample collected within a window of 5 minutes from the prescribed time point. From each sample, 20 μL of plasma was obtained and stored at dry ice temperatures for analysis. Analysis of each sample for the concentration of compound (1-1) was performed using a validated liquid chromatography tandem mass spectrometry (LC-MS/MS) method with a lower limit of quantitation of 0.5 ng/mL.

Pharmacokinetic parameters were calculated with the Phoenix WinNonlin software package (version 6.2.1, Pharsight Corp., CA, USA) with standard noncompartmental methods. The elimination phase half-life (t_{1/2}) was calculated by least-squares regression analysis of the terminal linear part of the log concentration-time curve. The area under the plasma concentration-time curve (AUC) was determined by use of the linear trapezoidal rule up to the last measurable concentration and thereafter by extrapolation of the terminal elimination phase to infinity. The mean residence time (MRT), representing the average amount of time a compound remains in a compartment or system, was calculated by extrapolating the drug concentration profile to infinity. The maximum plasma concentration (Cₘₐₓ) and the time to Cₘₐₓ (tₘₐₓ) were derived directly from the plasma concentration data. The tentative oral bioavailability (F) was calculated by dividing the dose normalised AUC after oral administration by the dose normalised AUC after intravenous administration, i.e. F=(AUC(oral)/Dose(oral))/(AUC(intravenous)/Dose(intravenous))] and is reported as percentage (%).

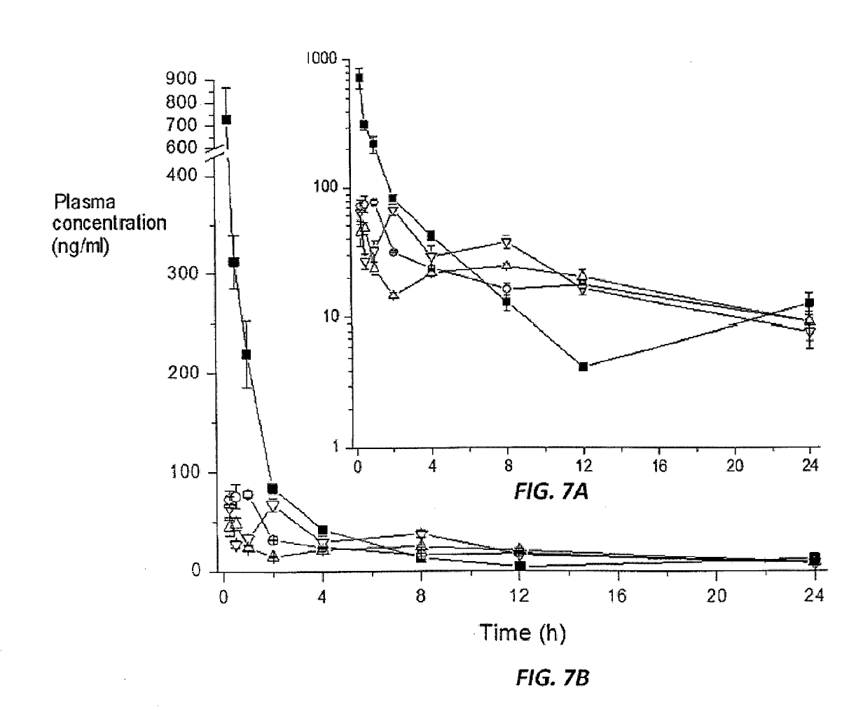

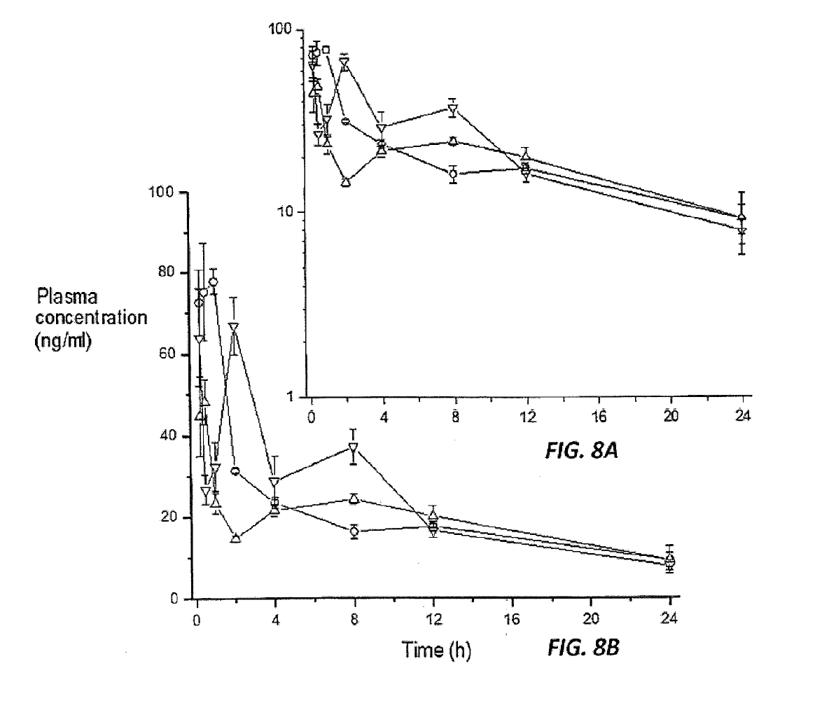

The pharmacokinetic parameters are given in Table 5, and the plasma concentration versus time plots are shown in FIGS. 7 and 8.

TABLE 5
Pharmacokinetic parameters of compound (1-1) after oral and intravenous
administrations. The values are an average from three animals.

| | | 1 mg/kg | 3 mg/kg | |
|---|---|---|---|---|
| Compound | Parameter | intravenous | oral | F (%) |
| Compound (1-1) | AUC (min*ng/ml) | 74698 | | |
| water:ethanol:PEG | Cₘₐₓ (ng/ml) | 730 | | |
| 400 (60:20:20) | Tₘₐₓ (hr) | 0.25 | | |
| | t_{1/2} (hr) 8.5 | 8.5 | | |
| | CI/F (ml/min/kg) | 13.4 | | |
| | MRT (hr) | 7.4 | | |
| 25% dispersion of | AUC (min*ng/ml) | | 39920 | 18 |
| compound (1-1) in | Cₘₐₓ (ng/ml) | | 77.9 | |
| PVP | Tₘₐₓ (hr) | | 1 | |
| | t_{1/2} (hr) 8.5 | | 13.8 | |
| | CI/F (ml/min/kg) | | 75.2 | |
| | MRT (hr) | | 18.0 | |
| 25% dispersion of | AUC (min*ng/ml) | | 35306 | 16 |
| compound (1-1) in | Cₘₐₓ (ng/ml) | | 48.3 | |
| HPMCAS-MG | Tₘₐₓ (hr) | | 0.5 | |
| | t_{1/2} (hr) 8.5 | | 11.0 | |
| | CI/F (ml/min/kg) | | 85.0 | |
| | MRT (hr) | | 17.1 | |
| 50% dispersion of | AUC (min*ng/ml) | | 40238 | 18 |
| compound (1-1) in | Cₘₐₓ (ng/ml) | | 67.0 | 9.5 |
| HPMCAS-MG | Tₘₐₓ (hr) | | 2 | |
| | t_{1/2} (hr) 8.5 | | 8.5 | |
| | CI/F (ml/min/kg) | | 74.6 | |
| | MRT (hr) | | 12.8 | |

### Example 5

### Preparation of Spray Dried Dispersions

Spray dried dispersions of compound (1-1) were prepared using five selected polymers: HPMCAS-MG (Shin Etsu Chemical Co., Ltd.), HPMCP-HP55 (Shin Etsu Chemical Co., Ltd.), PVP (ISP, a division of Ashland, Inc.), PVP-VA (BASF Corp.), and Eudragit L100-55 (Evonik Industries AG). All spray dried solutions were prepared at 25% and 50% by weight with each polymer. All solutions were prepared in acetone, with the exception of the PVP solutions, which were prepared in ethanol. For each solution, 1.0 g of solids (polymer and compound (1-1)) were prepared in 10 g of solvent. The solutions were spray dried using a Büchi B-290, PE-024 spray dryer with a 1.5 mm nozzle and a Büchi B-295, P-002 condenser. The spray dryer nozzle pressure was set to 80 psi, the target outlet temperature was set to 40° C., the chiller temperature was set to −20° C., the pump speed was set to 100%, and the aspirator setting was 100%. After spray drying, the solid dispersions were collected and dried overnight in a low temperature convection oven to remove residual solvents

### Example 6

### Stability with Humidity and Temperature

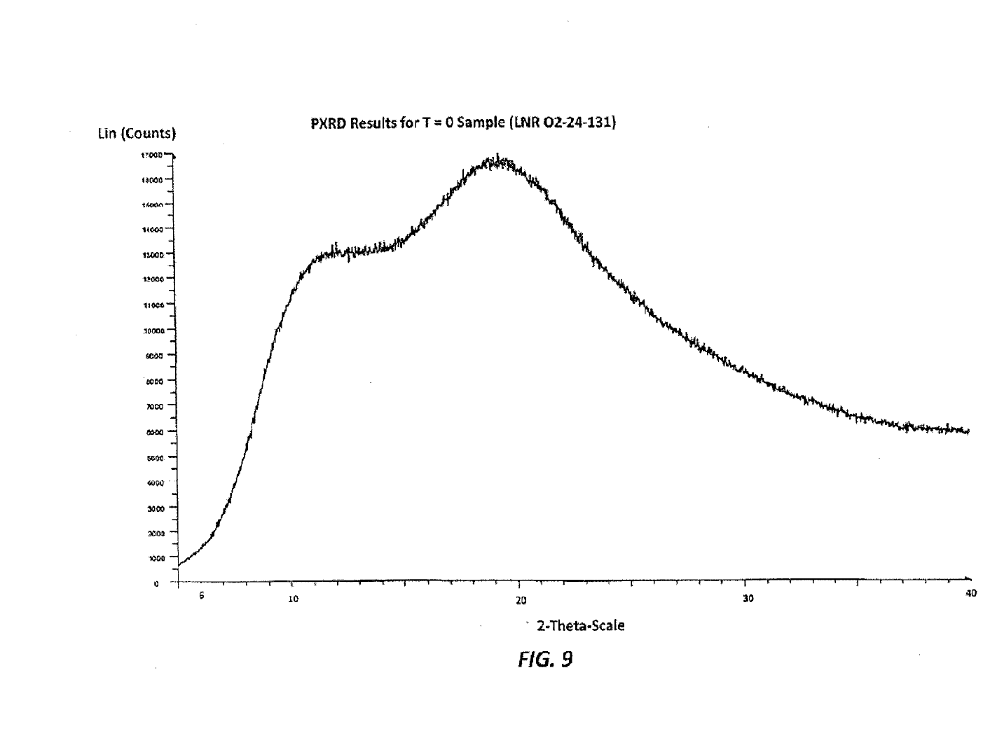

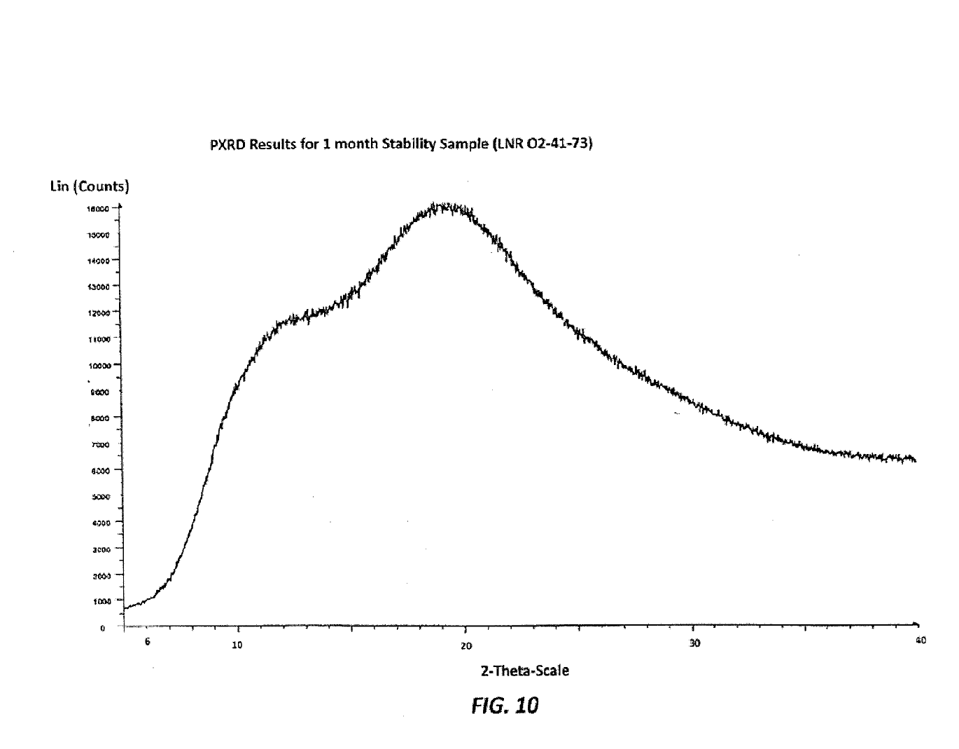

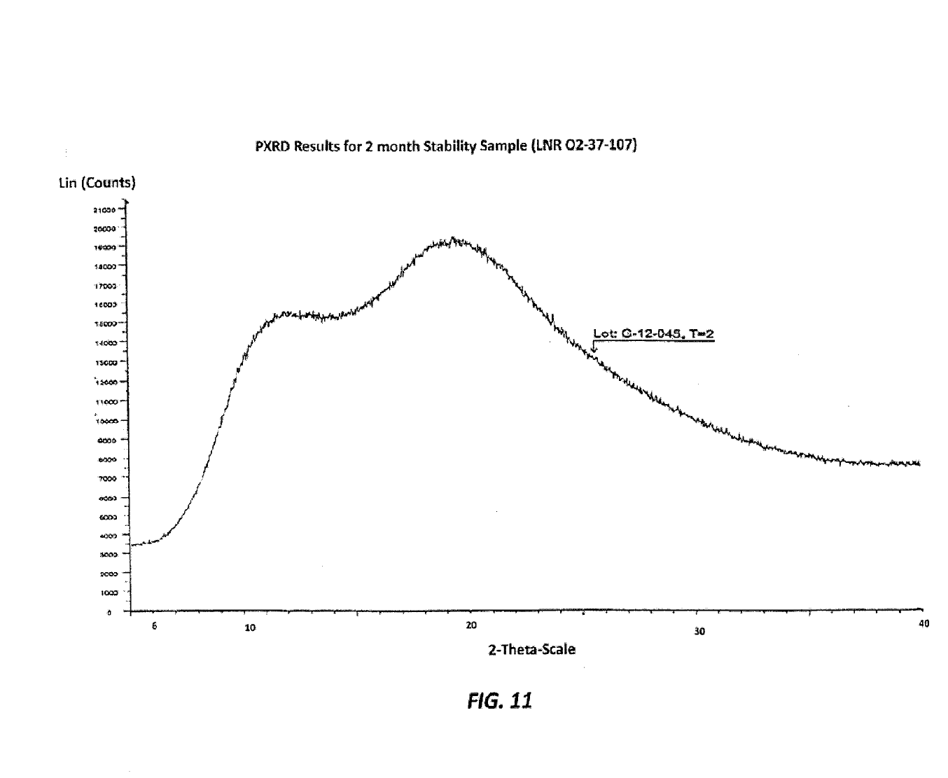

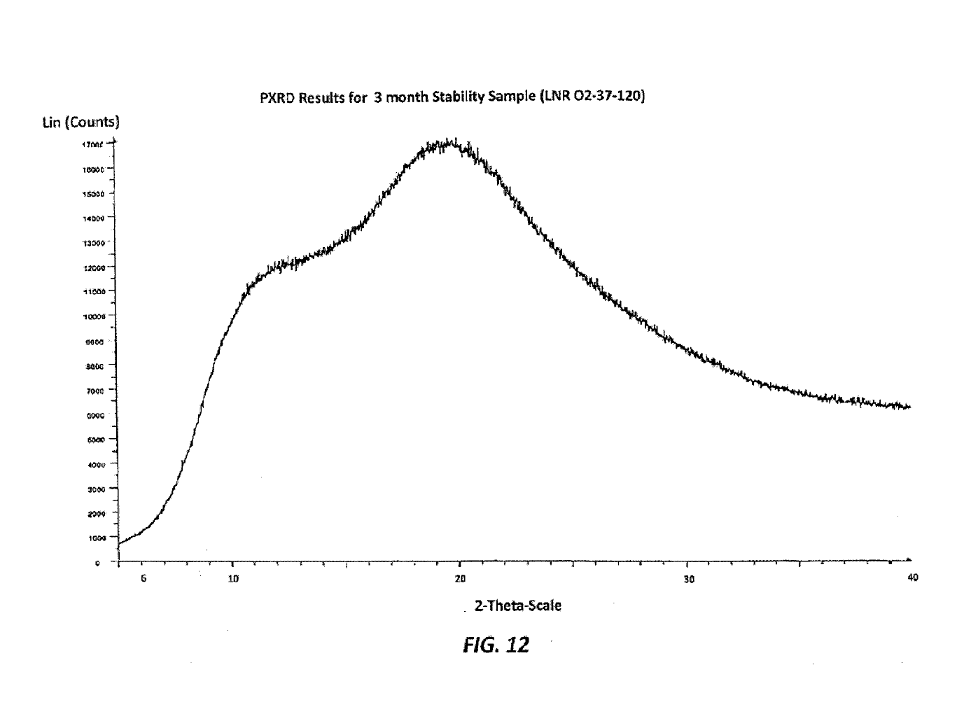

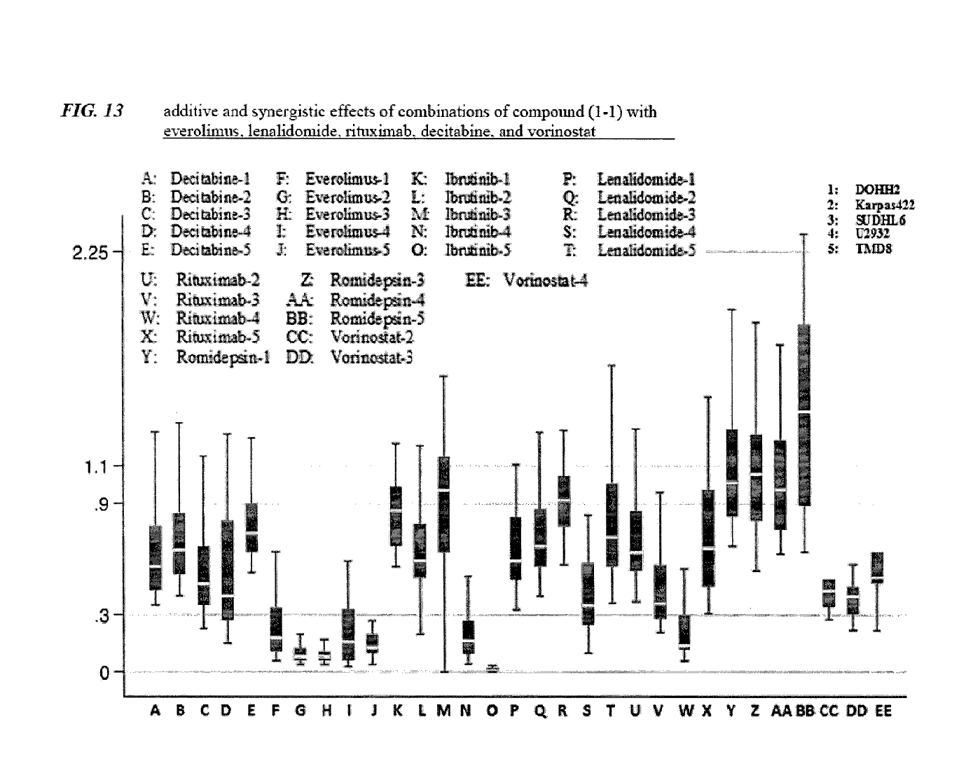

TABLE 6

| | | | | | T − 1 month | | T − 2 month | | T = 3 month | |
|---|---|---|---|---|---|---|---|---|---|---|
| | | Acceptance | | | (storage at 40° C./ | | (storage at 40° C./ | | (storage at 40° C./ | |
| Test | Procedure | Criteria | T = O (Initial) | | 75% RH) | | 75% RH) | | 75% RH) | |
| Appearance | AM-0002 | White to off- | Test Date/Ref: | | Test Date/Ref: | | Test Date/Ref: | | Test Date/Ref: | |
| | | white powder | 06 Aug. 2012/02-41-2 | | 24 Sep. 2012/ | | 24 Oct. 2012/ | | 17 Dec. 2012/ | |
| | | | White Powder | | 02-41-59 | | 02-37-106 | | 02-37-119 | |
| | | | | | White Powder | | White Powder | | White Powder | |
| Potency | AM-0028 | 45.0 · 55.0 wt % | Test Date/Ref: | | Test Date/Ref: | | Test Date/Ref: | | Test Date/Ref: | |
| (HPLC) | | | 25 Jul. 2012/ | | 25 Sep. 2012/ | | 24 Oct. 2012/ | | 29 Nov. 2012/ | |
| | | | 02-37-21 | | 4HI0 | | 02-37-1 05 | | 02-34-107 | |
| | | | 50.0 | | 49.4 | | 49.8 | | 49.2 | |
| Individual | AM-0029 | Report results | Test Date/Ref: | | Test Date/Ref: | | Test Date/Ref: | | Test Date/Ref: | |
| Related | | | 25 Jul. 2012/ | | 26 Sep. 2012/ | | 24 Oct. 2012/ | | 29 Nov. 2012/ | |
| Substances | | | 02-34-49 | | 02-41-64 | | 02-37-105 | | 02-34-107 | |
| (HPLC) | | | RRT | % Area | RRT | % Area | RRT | % Area | RRT | % Area |

| | | | | | |
|---|---|---|---|---|---|
| No reportable related | No reportable related | 0.68 | 0.06 | 0.68 | 0.07 |
| substances | substances | 0.77 | 0.06 | 0.77 | 0.09 |

| | | | | | | |
|---|---|---|---|---|---|---|
| Total Related | AM-0029 | Report results | Test Date/Ref: | Test Date/Ref: | Test Date/Ref: | Test Date/Ref: |
| Substances | | | 25 Jul. 2012/ | 26 Sep. 2012/ | 24 Oct. 2012/ | 29 Nov. 2012/ |
| (HPLC) | | | 02-34-49 | 02-41-64 | 02-37-105 | 02-34-107 |
| | | | No reportable | No reportable | 0.12% | 0.16% |
| | | | related substances | related substances | | |
| Water Content | AM-0030 | Report results | Test Date/Ref: | Test Date/Ref: | Test Date/Ref: | Test Date/Ref: |
| (KF) | USP <921> | (wt %) | 02 Aug. 2012/ | 27 Sep. 2012/ | 25 Oct. 2012 | 29 Nov. 2012/ |
| | | | 02-41-1 | 02-37-99 | 102-37-110 | 02-37-116 |
| | | | 1.52 | 2.53 | 2.70 | 3.43 |
| X-Ray Powder | USP <941> | Consistent with an | Test Date/Ref: | Test Date/Ref: | Test Date/Ref: | Test Date/Ref: |
| Diffraction | | amorphous form | 24 Jul. 2012/ | 01 Oct. 2012/ | 24 Oct. 2012/ | 17 Dec. 2012/ |
| (XRPD) | | | 02-24-131 | 02-41-73 | 02-37-107 | 02-37-120 |
| | | | Consistent with an | Consistent with an | Consistent with an | Consistent with an |
| | | | amorphous form | amorphous form | amorphous form | amorphous form |
| | | | See FIG. 9 | See FIG. 10 | See FIG. 11 | See FIG. 12 |
| Modulated | USP <891> | Report individual | Test Dale/Ref: | Test Date/Ref: | Test Date/Ref: | Test Date/Ref: |
| Differential | (n = 2 | and average glass | 24 Jul. 2012/ | 26 Sep. 2012/ | 24 Oct. 2012/ | 17 Dec. 2012/ |
| Scanning | replicates) | transtion | 02-24-130 | 02-37-98 | 02-37-108 | 02-37-121 |
| Calorimetry | | temperatures | Replicate 1 = | Replicate 1 = | Replicate 1 = | Replicate 1 = |
| (mDSC) | | (T_{g}, ° C.) | 134.30° C., | 134.65° C., | 135.35° C., | 134.36° C., |
| | | | Replicate 2 = | Replicate 2 = | Replicate 2 = | Replicate 2 = |
| | | | 134.23° C., | 134.43° C., | 134.93° C., | 137.16° C., |
| | | | Replicate 3 = | Average = | Average = | Average = |
| | | | 135.28° C., | 134.54° C. | 135.14° C. | 135.76° C. |
| | | | Average = | | | |
| | | | 134.60° C. | | | |

Spray dried dispersions of compound (1-1) in HPMCAS-MG were assessed for stability by exposure to moisture at elevated temperature. The glass transition temperature (Tg) as a function of relative humidity was determined at 75% relative humidity, 40° C. for 1, 2 and 3 months. The spray dried dispersion was stored in an LDPE bag inside a HDPE bottle to simulate bulk product packaging. The data is summarized in Table 6. At time zero, the Tg was 134° C., at 1 month the Tg was 134° C., at 2 months the Tg was 135° C. and at 3 months the Tg was 134° C. and only a single inflection point was observed for each measurement. X-ray diffraction patterns were also obtained for each sample. FIG. 9 illustrates a powder X-ray diffraction profile of solid dispersions of compound (1-1) in HPMCAS-MG at time zero of a stability test. FIGS. 10, 11 and 12 illustrate powder X-ray diffraction profiles of solid dispersions of compound (1-1) in HPMCAS-MG
after 1 month, 2 months and 3 months, respectively, after exposure at 40° C. and 75% relative humidity. The patterns did not show any diffraction lines associated with compound (1-1).

The patterns did not show any diffraction lines associated with compound (1-1).

### Example 7

### Pathways and Genes Affecting Response/Resistance to BET Bromodomain Inhibitors in Lymphomas

Methods:

Baseline gene expression profiles (GEP) were obtained in 38 cell lines [22 diffuse large B-cell lymphoma (DLBCL), 8 anaplastic large T-cell lymphoma, 4 mantle cell lymphoma, 3 splenic marginal zone lymphoma, 1 chronic lymphocytic leukemia] with Illumina HumanHT-12 v4 Expression BeadChip. Genetic and biologic information were collected from literature. GEP/IC50 correlation (ASH 2012; ICML 2013) was assessed by Pearson correlation. Associations in two-way tables were tested for statistical significance using either chi-square or Fisher exact test, as appropriate. Differential expression analysis was performed using LIMMA, followed by multiple test correction using the BH method. Enrichment of functionally-related genes was evaluated by GSEA.

Results:

Transcripts associated with resistance to compound (1-1) were significantly enriched of genes involved in cell cycle regulation, DNA repair, chromatin structure, early B-cell development, E2F/E2F2 target genes, IL6-dependent genes, and mRNA processing. Conversely, transcripts associated with compound (1-1) sensitivity were enriched of hypoxia-regulated genes, interferon target genes, STAT3 targets, and involved in glucose metabolism. Genes associated with compound (1-1) sensitivity included LDHA, PGK1 (glucose metabolism) and VEGFA (hypoxia), while BCL2L1/BCLXL, BIRC5/survivin (anti-apoptosis), ERCC1 (DNA repair), TAF1A and BRD7 (transcription regulation) were correlated with reduced sensitivity.

GEP identified 50 transcripts differentially expressed, including IL6, HCK, SGK1, MARCH1 and TRAFD1, between cells undergoing or not apoptosis after compound (1-1) exposure. GSEA showed significant enrichment of genes involved in IL-10 signaling pathway. While there was no association between response to compound (1-1)<500 nM and presence of translocated MYC, analysis of genetic and biologic features identified the ABC phenotype (P=0.008) and presence of concomitant somatic mutations in MYD88 and CD79B or CARD11 genes and wild type TP53 (P=0.027) as associated with apoptosis. Based on these observations and since mutated MYD88 interacts with BTK and MYD88/CD79B mutations have been associated with clinical responses with the BTK inhibitor ibrutinib, we evaluated compound (1-1) combination with this compound. Synergy was observed in particular in ABC-DLBCL with a median CI of 0.04 (range 0.02-0.1). The demonstrated down-regulation of the MYD88/JAK/STAT pathway after compound (1-1) treatment, as shown by additional GEP, highlighted the importance of this pathway for compound (1-1) activity.

### Example 8

### Pathways and Genes Affecting Response/Resistance to BET Bromodomain Inhibitors in Lymphomas

Methods:

3 germinal center B cell (GCB) DLBCL (DOHH2; Karpas422; and SUDHL6) and 2 activated B cell (ABC) DLBCL cell lines (U2932 and TMD8) were exposed to increasing doses of thienopyrazolodiazepine compound (1-1) alone or in combination with increasing doses of other drugs. The MTT assay was performed after 72 hours of exposure. Synergy was assessed by Chou-Talalay combination index (CI) with the Synergy R package: confidence interval (CI) <0.3, strong synergism; 0.3-0.9, synergism; 0.9-1.1, additive effect.

Baseline gene expression profiles (GEP) were obtained in 38 lymphoma cell lines, including 22 DLBCL with Illumina HumanHT-12 v4 Expression BeadChip. GEP before and after OTX015 treatment were done in 3 DLBCL cell lines, too. The relationship between GEP and IC50 values was assessed by Pearson correlation. LIMMA was used for differential expression analysis, followed by Benjamini-Hochberg multiple test correction, and GSEA to test for enrichment of functionally-related genes.

Results:

Strong synergism was observed with thienopyrazolodiazepine compound (1-1) combined with the mTOR inhibitor everolimus (median CI, 0.11; range 0.1-0.17) and with the BTK-inhibitor ibrutinib in ABC-cells (CI=0.04; 0.02-0.1). A synergistic effect was estimated for thienopyrazolodiazepine compound (1-1) in combinations with the class I and II HDAC-inhibitor vorinostat (CI=0.45; 0.31-0.56), the anti-CD20 moAb rituximab (CI=0.47; 0.37-0.54), the hypomethylating agent decitabine (CI=0.62; 0.56-0.66), and the immunomodulant lenalidomide (CI=0.66; 0.59-0.72). Thienopyrazolodiazepine compound (1-1) combinations with the class I HDAC inhibitor romidepsin (CI=1.08; 1-1.22) and with the chemotherapy agents bendamustine (CI=0.92; 0.83-1.1) and doxorubicin (CI=0.83; 0.71-0.96) presented a moderate additive effect. A stronger synergism was observed in ABC than in GCB DLBCL cells for ibrutinib (P<0.0001), lenalidomide (P=0.0001), and rituximab (P=0.007).

Data mining of GEP obtained at baseline across 38 lymphoma cell lines with known OTX015 IC50s and GEP changes observed after OTX015 exposure indicated the relevance of genes involved in MYD88/JAK/STAT pathway and glucose metabolism as possible explanations of the observed synergism of TOX015 with targeted agents, such as ibrutinib and everolimus.

### Example 9

### Analysis of the BET Bromodomain Inhibitor OTX015 and the NFKB, TLR, and JAK/STAT Pathways

Methods

Cell lines: 22 diffuse large B-cell lymphoma (DLBCL), 4 mantle cell lymphomas, 3 multiple myelomas, 3 splenic marginal zone lymphoma and 1 prolymphocytic leukemia. Anti-proliferative of OTX015 (OncoEthix SA, Switzerland) was assessed by MTT and its cytotoxic activity by Annexin V staining and gene expression profiling (GEP) with Illumina HumanHT-12 Expression BeadChips. Data mining was done with LIMMA, GSEA, Metacore.

Results

Compound (1-1) (500 nM, 72 h) showed cytostatic activity in 29/33 (88%) cell lines and apoptosis in 3/22 (14%). Mutations in genes coding for MYD88 and components of BCR (P=0.027), and ABC signaling phenotypes (P=0.008) were significantly associated with apoptosis induction. We performed GEP on 2 cell lines (SU-DHL-6, SU-DHL-2), treated with DMSO or OTX015 (500 nM) for 1, 2, 4, 8 or 12 hours. Most upregulated genes were histones. MYC target genes were highly significantly enriched among all Compound (1-1) regulated transcripts and MYC was the most frequently downregulated gene. Compound (1-1) also downregulated MYD88, IRAK1, TLR6, IL6, STAT3, and TNFRSF17, members of the NFKB, TLR and JAK/STAT pathways. NFKB target genes (IRF4, TNFAIP3 and BIRC3) were also downregulated (PCR). Immunoblotting and immunohistochemistry showed a reduction of transcriptionally active pSTAT3 in 2 ABC cell lines, and a reduction in nuclear localization of p50 (NFKB 1), indicating an inhibitory effect of OTX015 on the canonical NFKB pathway. Finally, IL10 and IL4 production was reduced after 24 hours OTX015 treatment.

### Example 9

### An Analysis of Gene Expression Profiles Before and after Exposure to BET Bromodomain Inhibitors

TABLE B

| | | | Multiple | | | | |
|---|---|---|---|---|---|---|---|
| | Diffuse | | myeloma, | | Multiple | | |
| | large B- | | Acute myeloid | | myeloma a | | B-cell acute |
| | cell | Lung | leukemia and | | Burkitt | Multiple | lymphoblastic |
| Disease | lymphoma¹ | adenocarcinoa³ | Neuroblasto³* | Neuroblastoa | lymphoma⁴ | myeloma⁵ | leukemia⁶ |
| Drug | OTX015 | JQ1 | JQ1 | JQ1 | JQ1 | JQ1 | JQ1 |
| Dose | 0.5 μM | 1 μM | Various | 1 μM | 0.5 μM | 0.5 μM | 0.5 μM |
| Time | 4-8 hrs. | 6 hrs. | Various | 24 hrs. | 4-8 hrs. | 24 hrs. | 8 hrs. |
| Platform | Illumina | Affymetrix | Affymetrix | Affymetrix | Affymetrix | Affymetrix | Affymetrix |
| | HumanHT- | GeneChip Exon | GeneChip | GeneChip | GeneChip | GeneChip | GeneChip |
| | 12 v4 | 1.0ST | Exon 1.0ST | PrimeView | Exon | Exon | Exon 1.0ST |
| | Expression | | | | 1.0ST | 1.0ST | |
| | BeadChip | | | | | | |
| Gene lists | Top 50 up, | Top 20 up, | 17 up, | Top 50 up, | Top 20 up, | Top 50 up, | Top 50 up, |
| | Top 50 | Top 20 down | 36 down* | Top 50 down | Top 20 down | Top 50 | Top 50 down |
| | down | | | | | down | |

*genes changing in common direction in the 3 cancers3*
OTX015: thienopyrazolodiazepine compound (1-1).
JQ1: (S)-tert-butyl 2-(4-(4-chloropheny1)-2,3,9-trimethyl-6H-thieno[3,2-f][1,2,4]triazolo[4,3-a][1,4]diazepin-6-yl)acetate.
¹Boi M, Bonetti P, Gaudio E, et al. “The BRD-inhibitor OTX015 is active in pre-clinical B-cell lymphoma models and affects relevant pathogenetic pathways”, Hematological Oncology (ICML Proceedings) 2013: in press.
²Lockwood WW, Zejnullahu K, Bradner JE, Varmus H. “Sensitivity of human lung adenocarcinoma cell lines to targeted inhibition of BET epigenetic signaling proteins”, Proc Natl Acad Sci USA 2012,109(47): 19408-19413.
³Puissant A, Frumm SM, Alexe G, et al. “Targeting MYCN in Neuroblastoma by BET Bromodomain Inhibition”, Cancer Discov. 2013.
⁴Mertz JA, Conery AR, Bryant BM, et al. “Targeting MYC dependence in cancer by inhibiting BET bromodomains”, Proc Natl Acad Sci USA 2011, 108(40): 16669-16674.
⁵Delmore JE, Issa GC, Lemieux ME, et al. “BET bromodomain inhibition as a therapeutic strategy to target c-Myc”, Cell 2011,146(6): 904-917.
⁶Ott CJ, Kopp N, Bird L, et al. “BET bromodomain inhibition targets both c-Myc and IL7R in high-risk acute lymphoblastic leukemia”, Blood 2012, 120(14): 2843-2852.

TABLE C
Reported gene expression signatures

| | | Lung | MM, AML, | | MM | | |
|---|---|---|---|---|---|---|---|
| | | adeno- | Neuro- | Neuro | and | | |
| | DLBCL¹ | carcinoma 2 | blastoma 3* | blastoma³ | BL⁴ | MM⁵ | B-ALL⁶ |
| Down** | | | | | | | |
| 1. | ADORA2A | ADORA2B | ADAT2 | ADORA2B | ADAT2 | ABCC4 | ACSL5 |
| 2. | AICDA | ARL14 | ALG14 | AEBP1 | ALKBH8 | ABLIM1 | ALKBH8 |
| 3. | ARHGAP25 | CLCF1 | ALKBH8 | ANK3 | AMKRD37 | ACSL5 | BST2 |
| 4. | BATF | FOSL1 | BDH1 | ARHGAP23 | C1orf107 | ACSM3 | C17orf87 |
| 5. | BBOX1 | GPR87 | C12orf24 | AS3MT | C1orf163 | ADAT2 | CARD17 |
| 6. | BCL6 | HAS2 | C1orf163 | ASB13 | CCR1 | ALDH1B1 | CCDC26 |
| 7. | BID | IL7R | C1orf31 | BATF3 | CD180 | AMPD1 | CCDC86 |
| 8. | BRIX1 | LOC388022 | CCDC58 | C14orf1 | CD48 | BDH1 | CCL2 |
| 9. | C12ORF24 | LOC728377 | CLPP | C18orf55 | CXCL10 | BTN3A2 | CD72 |
| 10. | CCDC86 | LYPD1 /// | E2F8 | C1orf31 | FJX1 | CCR1 | CMAH |
| | | GPR39 | | | | | |
| 11. | COBL | MDM2 | FAR2 | C5orf43 | MYB | CDC25A | DFNA5 |
| 12. | CUTC | MMACHC | FKBP4 | CC2D2A | MYC | DERL3 | DHX33 |
| 13. | DCUN1D5 | MTL5 | GALC | CHRM1 | PRDM10 | FADS1 | DOK3 |
| 14. | DDX21 | NEXN | GPATCH4 | DLAT | PTAFR | FKBP11 | FAIM3 |
| 15. | DHRS9 | RUNX2 | GTF3C6 | FAM101A | RGS1 | GALNT14 | FLJ21272 |
| 16. | EBI2 | SEMA4B | IFRD2 | GTF3C6 | SLAMF7 | GTF3C6 | GJB2 |
| 17. | GAPT | SEMA4C | IRAK1 | HDAC9 | SLC16A6 | HBD | GLDC |
| 18. | HNRNPD | SLITRK6 | MAGOHB | HOXC8 | TNFRSF17 | KAT2A | GLIPR1 |
| 19. | IL21R | TRAF1 | MRTO4 | ITPRIPL2 | ZMYND8 | KCNA3 | IL7R |
| 20. | KDELC2 | TSKU | MTHFD1L | JAM2 | ZNF487 | KCNQ5 | LILRA2 |
| 21. | LAT2 | | MTMR2 | LOC1001307 | | MANEAL | LOC728175 |
| | | | | 76 | | | |
| 22. | LRMP | | NOP16 | LRP8 | | MAP1D | MLKL |
| 23. | LRRC33 | | NR2C2AP | LTV1 | | MAP4K1 | MPO |
| 24. | LYSMD2 | | OBFC2B | MAPK3 | | MGC29506 | MTHFD1L |
| 25. | MLKL | | PEMT | MRPL11 | | MMACHC | MTMR2 |
| 26. | MYB | | POLE2 | MRPL15 | | MORCI | MYC |
| 27. | MYC | | PPRC1 | MTHFD1L | | MTHFD1L | NCF2 |
| 28. | NAPSB | | RAB7L1 | NOP16 | | MTMR2 | NEXN |
| 29. | OAS2 | | RNASEH2B | OAF | | MYB | NIPAL2 |
| 30. | P2RY8 | | SFXN4 | PA2G4 | | MYC | NME1 |
| 31. | PHF15 | | TMEM126A | PLIN3 | | NAV1 | NOG |
| 32. | PLD6 | | TSGA14 | PON2 | | NME1 | PECAM1 |
| 33. | PTPN6 | | TTC27 | RAB33A | | POLE2 | PEMT |
| 34. | PVRIG | | TYRO3 | RAB7L1 | | POLR3G | PLAC8 |
| 35. | RASGRP3 | | UBXN8 | RAC3HEA | | PTPN22 | POLR1B |
| 36. | RRS1 | | UNG | RGS19 | | RAI14 | PPRC1 |
| 37. | SERPINA9 | | | RNF157 | | RNF125 | PSAT1 |
| 38. | SFRS3 | | | SLC18A1 | | RRS1 | PTPN22 |
| 39. | SGK1 | | | SLC5A6 | | SFXN4 | PVRIG |
| 40. | SLC25A43 | | | SORBS3 | | SLC16A9 | RCN1 |
| 41. | SLC2A5 | | | SULF2 | | SLC19A1 | RRS1 |
| 42. | ST6GAL1 | | | TBL1XR1 | | SLC38A5 | SFXN4 |
| 43. | STAMBPL1 | | | TBL2 | | SLC7A2 | SLC22A16 |
| 44. | TNFRSF17 | | | TFAP2B | | SORD | SLC38A5 |
| 45. | TNS3 | | | TH | | SRM | SLC7A11 |
| 46. | TP63 | | | TOMM40L | | TTC27 | STS |
| 47. | TRIP6 | | | TR2 | | TYRO3 | THBS1 |
| 48. | TSEN2 | | | UBL4A | | UNQ3104 | TXNDC3 |
| 49. | TSGA14 | | | UTRN | | XTP3TPA | VAMP8 |
| 50. | UBE2J1 | | | ZMYND8 | | ZNF485 | ZNF487P |
| Up** | | | | | | | |
| 1. | ADARB1 | ARRDC4 | AP1G2 | AP1G2 | ATP1B1 | APOLD1 | AASS |
| 2. | BRD2 | C7orf53 | BNIP3L | ARL3 | C7orf53 | BMPR2 | ACBD7 |
| 3. | C12ORF34 | CCNE2 | C1orf63 | BBS4 | CSRNP2 | BNIP3L | APLP2 |
| 4. | CCL5 | CTGF | CSRNP2 | C17orf108 | HEXIM1 | C13ORF31 | ARHGAP26 |
| 5. | DCXR | DUSP1 | DAAM1 | C19orf30 | HIST1H2AG | C1ORF26 | ARSK |
| 6. | DHRS2 | GCLC | FGD6 | C19orf63 | HIST1H2BD | C1ORF63 | BTD |
| 7. | H1FX | HIST1H1T | HEXIM1 | C1orf63 | HIST1H2BJ | C9ORF95 | BVES |
| 8. | H2AFJK | HIST1H2BJ | HIST2H4A | C5orf55 | HIST1H2B | CALCOCO1 | CAPRIN2 |
| 9. | HES6 | HIST1H4H | ITFG3 | D2HGDH | HIST2H2BE | CLDN12 | CCNYL1 |
| 10. | HIST1H1C | HIST2H2BE | KLHL24 | DCXR | HIST2H2BF | CNTN5 | CDKL5 |
| 11. | HIST1H2AC | HIST2H2BF | PAG1 | DNAJC1 | NXF1 | DNAJC28 | CPEB4 |
| 12. | HIST1H2BD | HS6ST1 | PNRC1 | FAM164A | OR2B6 | DNM3 | CSRNP2 |
| 13. | HIST1H2BG | LOC93622 | SERPINI1 | FILIP1L | POLR2A | DOPEY2 | DCXR |
| 14. | HIST1H2BJ | OR2B6 | STX7 | GCH1 | SAT1 | HEXIM1 | DNAJB4 |
| 15. | HIST1H2BK | PAG1 | TP53INP1 | GCLC | SESN3 | HHLA3 | DNAJC1 |
| 16. | HIST1H3D | SESN3 | TUFT1 | GDF11 | SLFN5 | HIST2H2BE | EFR3B |
| 17. | HIST1H3F | SLC10A5 | ZSWIM6 | HEXIM1 | TMEM2 | HIST2H4A | EPHX1 |
| 18. | HIST2H2AA3 | SLC6A8 /// | | HIST | TUBA1A | ITFG3 | FAM46C |
| | | SLC6A10P | | 1H2AC | | | |
| 19. | HIST2H2AA4 | TOB1 | | HIST1H2AE | TXNIP | JARID1B | FGD6 |
| 20. | HIST2H2AC | ZNF14 | | HIST1H2AG | WDR47 | JHDM1D | FLJ38109 |
| 21. | HIST2H2BE | | | HIST1H2BC | | KIAA0825 | GLCE |
| 22. | HIST2H4A | | | HIST1H2BK | | KIAA0913 | GLIPR2 |
| 23. | IRF7 | | | HIST2H2AA3 | | KLHL24 | HEXIM1 |
| 24. | KIAA1683 | | | HIST2H2BC | | LGALS1 | HIST1H2BD |
| 25. | LRCH4 | | | INPP4A | | LMNA | HIST1H2BJ |
| 26. | MKNK2 | | | KCTD21 | | LYST | HIST2H2BE |
| 27. | MT1A | | | LOC728392 | | MAP2 | HIST2H2BF |
| 28. | MT1E | | | LOC729991 | | NFKBIZ | LYST |
| 29. | MT1G | | | MYH9 | | OR2B6 | MXD1 |
| 30. | MT1X | | | NEU1 | | PAG1 | NDRG1 |
| 31. | MT2A | | | OS9 | | PNPLA8 | NEU1 |
| 32. | MTE | | | PAG1 | | RNF19B | NMT2 |
| 33. | MXD4 | | | PCDH17 | | SAT1 | OR2L3 |
| 34. | NEU1 | | | PCMTD1 | | SATB1 | OR52H1 |
| 35. | NXF1 | | | PIM1 | | SCN9A | PELI1 |
| 36. | OCEL1 | | | PJA2 | | SEPP1 | PPP1R13B |
| 37. | PDLIM7 | | | POLG | | SERPINI1 | PRKAR2B |
| 38. | PNPLA2 | | | PPP3CB | | SESN3 | PTPN12 |
| 39. | POLR2A | | | RALGAPA1 | | SLC12A6 | SAT1 |
| 40. | PPP1R13B | | | RPL12 | | SQSTM1 | SESN3 |
| 41. | RGS2 | | | SCARNA20 | | STAT2 | SH3PXD2B |
| 42. | SERTAD1 | | | SDCBP | | SYT11 | SLC44A1 |
| 43. | SNORD3A | | | SERPINI1 | | TMEM2 | TARSL2 |
| 44. | SNORD3D | | | SERTAD1 | | USP11 | TESK2 |
| 45. | SPTAN1 | | | TAX1BP3 | | WDR47 | TM7SF2 |
| 46. | TMEM175 | | | THAP8 | | YPEL1 | TMEM50B |
| 47. | TNFSF9 | | | TMEFF2 | | YPEL5 | TRIM62 |
| 48. | TUBB2C | | | TMEM8A | | ZFP36 | USP53 |
| 49. | TUBB3 | | | TUFT1 | | ZFYVE1 | VCL |
| 50. | TUBB4Q | | | ZNF480 | | ZSWIM6 | WDR47 |

Legend for Table G:
DLBCL: Diffuse large B-cell lymphoma;
MM: Multiple myeloma;
AML: Acute myeloid leukemia;
BL: Burkitt lymphoma;
B-ALL: B-cell acute lymphoblastic leukemia.
**sorted in alphabetical order
*as reported common to MM, AML and Neuroblastoma3

### Example 9

### Genes that are Down-Regulated by Bet Bromodomain Inhibitors in More than Two of the Seven Gene-Lists Above-Reported (See Refs. 1-6 Above)

| | Number of studies in which |
|---|---|
| Gene | the gene is reported as changing |
| MTHFD1L | 4/7 |
| MYC | 4/7 |
| ADAT2 | 3/7 |
| ALKBH8 | 3/7 |
| GTF3C6 | 3/7 |
| MTMR2 | 3/7 |
| MYB | 3/7 |
| RRS1 | 3/7 |
| SFXN4 | 3/7 |

### Example 10

### Genes that are Up-Regulated by Bet Bromodomain Inhibitors in More than Two of the Seven Gene-Lists Above-Reported (See Refs. 1-6 Above)

| | Number of studies in which |
|---|---|
| Gene | the gene is reported as changing |
| HEXIM1 | 5/7 |
| HIST2H2BE | 5/7 |
| HIST1H2BJ | 4/7 |
| SESN3 | 4/7 |
| C1orf63 | 3/7 |
| CSRNP2 | 3/7 |
| HIST1H2BD | 3/7 |
| HIST1H2BK | 3/7 |
| HIST2H2BF | 3/7 |
| HIST2H4A | 3/7 |
| NEU1 | 3/7 |
| OR2B6 | 3/7 |
| PAG1 | 3/7 |
| SAT1 | 3/7 |
| SERPINI1 | 3/7 |
| WDR47 | 3/7 |

It will be appreciated by those skilled in the art that changes could be made to the exemplary embodiments shown and described above without departing from the broad inventive concept thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and features of the disclosed embodiments may be combined. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the method does not rely on the particular order of steps set forth herein, the particular order of the steps should not be construed as limitation on the claims. The claims directed to the method of the present invention should not be limited to the performance of their steps in the order written, and one skilled in the art can readily appreciate that the steps may be varied and still remain within the spirit and scope of the present invention.

1. A method of treating diffuse large B-cell lymphoma comprising administering to a patient a pharmaceutically acceptable amount of a composition comprising a thienotriazolodiazepine compound, said thienotriazolodiazepine compound being represented by the following Formula (1): 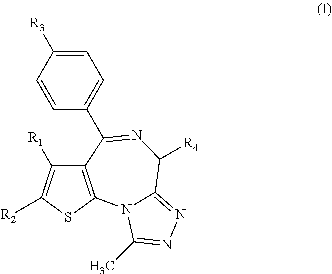
wherein R₁ is alkyl having a carbon number of 1-4, R₂ is a hydrogen atom; a halogen atom; or alkyl having a carbon number of 1-4 optionally substituted by a halogen atom or a hydroxyl group, R₃ is a halogen atom; phenyl optionally substituted by a halogen atom, alkyl having a carbon number of 1-4, alkoxy having a carbon number of 1-4 or cyano; —NR₅—(CH₂)ₘ—R₆ wherein R₅ is a hydrogen atom or alkyl having a carbon number of 1-4, m is an integer of 0-4, and R₆ is phenyl or pyridyl optionally substituted by a halogen atom; or —NR₇—CO—(CH₂)ₙ—R₈ wherein R₇ is a hydrogen atom or alkyl having a carbon number of 1-4, n is an integer of 0-2, and R₈ is phenyl or pyridyl optionally substituted by a halogen atom, and R₄ is —(CH₂)ₐ—CO—NH—R₉ wherein a is an integer of 1-4, and R₉ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR₁₀ wherein b is an integer of 1-4, and R₁₀ is alkyl having a carbon number of 1-4, or a pharmaceutically acceptable salt thereof or a hydrate or solvate thereof, wherein the patient has activated B-cell diffuse large B-cell lymphoma.

2. The method of claim 1 wherein the thienotriazolodiazepine compound represented by Formula 1 is independently selected from the group consisting of:
(i) (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide or a dihydrate thereof, (ii) methyl (S)-{4-(3′-cyanobiphenyl-4-yl)-2,3,9-trimethyl-6H-thieno[3,2-f][1,2,4]tri-azolo[4,3-a][1,4]diazepin-6-yl}acetate, (iii) methyl (S)-{2,3,9-trimethyl-4-(4-phenylaminophenyl)-6H-thieno[3,2-f][1,2,4]triaz-olo[4,3-a][1,4]diazepin-6-yl}acetate; and (iv) methyl (S)-{2,3,9-trimethyl-4-[4-(3-phenylpropionylamino)phenyl]-6H-thieno[3,2-f-][1,2,4]triazolo[4,3-a][1,4]diazepin-6-yl}acetate.

3. The method of claim 2, wherein the thienotriazolodiazepine compound is (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide dihydrate.

4. The method according to claim 3, wherein the thienotriazolodiazepine compound is formed as a solid dispersion comprising an amorphous thienotriazolodiazepine compound of the Formula (1) or a pharmaceutically acceptable salt thereof or a hydrate thereof; and a pharmaceutically acceptable polymer.

5. The method according to claim 4, wherein the solid dispersion exhibits an X-ray powder diffraction pattern substantially free of diffraction lines associated with crystalline thienotriazolodiazepine compound of Formula (1).

6. The method according to claim 5, wherein the solid dispersion exhibits a single glass transition temperature (Tg) inflection point ranging from about 130° C. to about 140° C.

7. The method according to claim 6, wherein the pharmaceutically acceptable polymer is hydroxypropylmethylcellulose acetate succinate having a thienotriazolodiazepine compound to hydroxypropylmethylcellulose acetate succinate (HPMCAS), weight ratio of 1:3 to 1:1.

8. The method according to claim 7, wherein the activated B-cell diffuse large B-cell lymphoma has concomitant somatic mutations in one or more of MYD88 gene, CD79B gene, CARD11 gene or wild type TP53 gene.

9. The method according to claim 8, wherein the compound represented by Formula (1) down regulates expression of one or more genes of MYD88 gene, IRAK1 gene, TLR6 gene, IL6 gene, STAT3 gene, and TNFRSF17 gene.

10. The method according to claim 9, wherein the compound represented by Formula (1) down regulates expression of one or more genes involved in the NFKB pathway, said genes selected from IRF4, TNFAIP3 and BIRC3.

11. A method of treating diffuse large B-cell lymphoma comprising administering to a patient a pharmaceutically acceptable amount of a composition comprising a thienotriazolodiazepine compound, said thienotriazolodiazepine compound being represented by the following Formula (1): 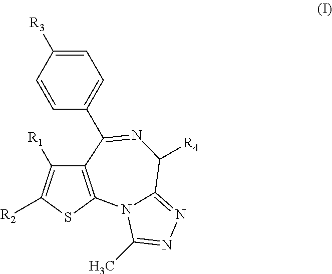
wherein R₁ is alkyl having a carbon number of 1-4, R₂ is a hydrogen atom; a halogen atom; or alkyl having a carbon number of 1-4 optionally substituted by a halogen atom or a hydroxyl group, R₃ is a halogen atom; phenyl optionally substituted by a halogen atom, alkyl having a carbon number of 1-4, alkoxy having a carbon number of 1-4 or cyano; —NR₅—(CH₂)ₘ—R₆ wherein R₅ is a hydrogen atom or alkyl having a carbon number of 1-4, m is an integer of 0-4, and R₆ is phenyl or pyridyl optionally substituted by a halogen atom; or —NR₇—CO—(CH₂)ₙ—R₈ wherein R₇ is a hydrogen atom or alkyl having a carbon number of 1-4, n is an integer of 0-2, and R₈ is phenyl or pyridyl optionally substituted by a halogen atom, and R₄ is —(CH₂)ₐ—CO—NH—R₉ wherein a is an integer of 1-4, and R₉ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR₁₀ wherein b is an integer of 1-4, and R₁₀ is alkyl having a carbon number of 1-4, or a pharmaceutically acceptable salt thereof or a hydrate or solvate thereof; wherein the thienotriazolodiazepine compound is formed as a solid dispersion comprising an amorphous thienotriazolodiazepine compound of the Formula (1) or a pharmaceutically acceptable salt thereof or a hydrate thereof, and a pharmaceutically acceptable polymer.

12. The method of claim 11 wherein the thienotriazolodiazepine compound represented by Formula 1 is independently selected from the group consisting of:
(i) (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide or a dihydrate thereof, (ii) methyl (S)-{4-(3′-cyanobiphenyl-4-yl)-2,3,9-trimethyl-6H-thieno[3,2-f][1,2,4]tri-azolo[4,3-a][1,4]diazepin-6-yl}acetate, (iii) methyl (S)-{2,3,9-trimethyl-4-(4-phenylaminophenyl)-6H-thieno[3,2-f][1,2,4]triaz-olo[4,3-a][1,4]diazepin-6-yl}acetate; and (iv) methyl (S)-{2,3,9-trimethyl-4-[4-(3-phenylpropionylamino)phenyl]-6H-thieno[3,2-f-][1,2,4]triazolo[4,3-a][1,4]diazepin-6-yl}acetate.

13. The method of claim 12, wherein the thienotriazolodiazepine compound is (5)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide dihydrate.

14. The method according to claim 13, wherein the solid dispersion exhibits an X-ray powder diffraction pattern substantially free of diffraction lines associated with crystalline thienotriazolodiazepine compound of Formula (1).

15. The method according to claim 14, wherein the solid dispersion exhibits a single glass transition temperature (Tg) inflection point ranging from about 130° C. to about 140° C.

16. The method according to claim 15, wherein the pharmaceutically acceptable polymer is hydroxypropylmethylcellulose acetate succinate having a thienotriazolodiazepine compound to hydroxypropylmethylcellulose acetate succinate (HPMCAS), weight ratio of 1:3 to 1:1.

17. The method according to claim 16, wherein the compound represented by Formula (1) down regulates expression of one or more genes of MYD88 gene, IRAK1 gene, TLR6 gene, IL6 gene, STAT3 gene, and TNFRSF17 gene.

18. The method according to claim 17, wherein the compound represented by Formula (1) down regulates expression of one or more genes involved in the NFKB pathway, said genes selected from IRF4, TNFAIP3 and BIRC3.

19. The method according to claim 18, wherein the patient has activated B-cell diffuse large B-cell lymphoma.

20. The method according to claim 19, wherein the activated B-cell diffuse large B-cell lymphoma has concomitant somatic mutations in one or more of MYD88 gene, CD79B gene, CARD11 gene or wild type TP53 gene.
